(19) United States
(12) Patent Application Publication
(10) Pub. No.: US 2011/0112121 A1
(43) Pub. Date: May 12, 2011

(54) Pharmaceutical Compositions and Solid Forms

(76) Inventors: Joerg BERGHAUSEN, Basel (CH); Claire HAUG, Basel (CH); Michael HERBIG, Basel (CH); Bin HU, East Hanover, NJ (US); Stéphane JONAT, Basel (CH); Rajender LELETI, East Hanover, NJ (US); Josef Gottfried MEINGASSNER, Vienna (AT); Stéphanie MONNIER, Cedex Compiegne (FR); Matthias NAPP, Basel (CH); Mahavir PRASHAD, East Hanover, NJ (US); Anton STÜTZ, Wien (AT); Ranjit THAKUR, Basel (CH)

(21) Appl. No.: 12/827,218

(22) Filed: Jun. 30, 2010

Related U.S. Application Data
(60) Provisional application No. 61/223,269, filed on Jul. 6, 2009.

(51) Int. Cl. A61K 31/505; A61P 17/00; A61P 17/06; C07D 239/02

(52) U.S. Cl. 514/269; 544/319

(57) ABSTRACT

The present invention relates to pharmaceutical compositions of 6-(6-hydroxymethyl-pyrimidin-4-yloxy)-naphthalene-1-carboxylic acid (3-trifluoromethyl-phenyl)-amide, to the use of 6-(6-hydroxymethyl-pyrimidin-4-yloxy)-naphthalene-1-carboxylic acid (3-trifluoromethyl-phenyl)-amide and compositions of 6-(6-hydroxymethyl-pyrimidin-4-yloxy)-naphthalene-1-carboxylic acid (3-trifluoromethyl-phenyl)-amide in therapeutic applications, especially indications with a dysregulation/overexpression of VEFG, (neo)-vascularisation and VEGF driven angiogenesis and to methods for manufacturing such compositions, the invention further relates to specific forms of 6-(6-hydroxymethyl-pyrimidin-4-yloxy)-naphthalene-1-carboxylic acid (3-trifluoromethyl-phenyl)-amide and to the manufacturing and use of such forms. The present invention also relates to a new process to produce 6-(6-hydroxymethyl-pyrimidin-4-yloxy)-naphthalene-1-carboxylic acid (3-trifluoromethyl-phenyl)-amide.

### FIELD OF THE INVENTION

The present invention relates to pharmaceutical compositions of 6-(6-hydroxymethyl-pyrimidin-4-yloxy)-naphthalene-1-carboxylic acid (3-trifluoromethyl-phenyl)-amide, to the use of 6-(6-hydroxymethyl-pyrimidin-4-yloxy)-naphthalene-1-carboxylic acid (3-trifluoromethyl-phenyl)-amide and compositions of 6-(6-hydroxymethyl-pyrimidin-4-yloxy)-naphthalene-1-carboxylic acid (3-trifluoromethyl-phenyl)-amide in therapeutic applications, especially indications with a dysregulation/overexpression of VEFG, (neo)-vascularisation and VEGF driven angiogenesis and to methods for manufacturing such compositions, the invention further relates to specific forms of 6-(6-hydroxymethyl-pyrimidin-4-yloxy)-naphthalene-1-carboxylic acid (3-trifluoromethyl-phenyl)-amide and to the manufacturing and use of such forms. The present invention also relates to a new process to produce 6-(6-hydroxymethyl-pyrimidin-4-yloxy)-naphthalene-1-carboxylic acid (3-trifluoromethyl-phenyl)-amide.

### BACKGROUND OF THE INVENTION

WO 2006/059234 describes certain naphthalene-1-carboxylic acid derivatives, such as 6-(6-hydroxymethyl-pyrimidin-4-yloxy)-naphthalene-1-carboxylic acid (3-trifluoromethyl-phenyl)-amide, a process to produce these derivatives and various pharmaceutical uses thereof. Further, this document suggests oral administration of such derivatives and very generally discloses pharmaceutical compositions in unit dosage form, such as capsules. WO 2006/059234 also describes that the naphthalene-1-carboxylic acid derivatives show inhibition of protein kinases especially the Vascular Endothelial Growth Factor Receptors (VEGF-Rs) such as in particular VEGF-R2.

WO 2007/031265 describes certain topical compositions comprising naphthalene-1-carboxylic acid derivatives and oleyl alcohol as a penetration enhancer; it also describes various pharmaceutical uses of such compositions.

There is still a need for the provision of agents with therapeutic efficacy in the diseases/disorders with a dysregulation/overexpression of VEFG, (neo)-vascularisation, VEGF driven angiogenesis and inflammation.

Rosacea is a common, chronic and progressive facial skin disorder. It mainly affects the central part of the face and is characterized by redness of the face or hot flushes. Rosacea is characterized by erythema, papules, pustules and telangiectasia (Wilkin J, Dahl M, Detmar, M, Drake L, Liang M H, Odom R, Powell F. Standard grading system for rosacea: Report of the National Rosacea Society Expert Committee on the Classification and Staging of Rosacea. J Am Acad Dermatol 2004 June; 61(6):907-12).

This disorder of the skin occurs most often between the ages of 25 and 70, and is generally more common in women, however, serious cases have been observed in men. Rosacea, in mild form (erythematotelangiectatic rosecea), brings about a slight flushing of the nose and cheeks and, in some cases, the forehead and chin. However, in more severe form (papulopustular rosacea) persistent central facial erythema with transient papules or pustules or both is observed. In another severe form (phymatous rosacea) thickening of the skin, irregular surface nodularities and enlargement is observed. Roseacea is also observed to affect the eye and eyelid. There is also a rare complication of rosacea, known as Morbihan disease, which is characterized by persistent lymphoedema on the upper half of the face, occurring during the chronic clinical course of rosacea (T. Nagasaka, T. Koyama, K. Matsumura, K. R. Chen. Persistent lymphoedema in Morbihan disease: formation of perilymphatic epithelioid cell granulomas as a possible pathogenesis. Clin Exp Dermat 2008, 33(6), 764-767).

Expression of VEGF is increased in the lesional skin in rosacea. (Gomaa A H, Yaar M, Eyada M M, Bhawan J. Lymphangiogenesis and angiogenesis in non-phymatous rosacea. J Cutan Pathol. 2007 October; 34(10):748-53; Laquer V, Hoang V, Nguyen A, Kelly K M. Angiogenesis in cutaneous disease: Part II. J Am Acad Dermatol 2009 December; 61(6):945-58).

On account of the multi-factor aspect of rosacea, there is a need for an effective treatment that is without risk for the patient associated with these treatments.

It is desirable to identify compositions, and uses of these compositions as well as new specific forms of compounds that may improve efficiency, bioavailability, stability and/or acceptance by the patient, and methods of manufacturing that may improve efficiency, number of steps, yield, cost of goods, safety profile, selectivity and reaction times.

These objectives are achieved by providing a composition and compound as defined herein, by providing the compound and composition thereof for use in diseases, particular for the treatment of dermatological diseases, as defined herein and by providing a process to produce the composition and the compound as defined herein.

Further aspects of the invention are disclosed in the specification and independent claims, preferred embodiments are disclosed in the specification and the dependent claims.

### SUMMARY OF THE INVENTION

The invention provides in its broadest sense topical pharmaceutical compositions containing the compound 6-(6-hydroxymethyl-pyrimidin-4-yloxy)-naphthalene-1-carboxylic acid (3-trifluoromethyl-phenyl)-amide:

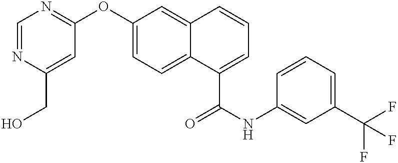

as agent of the invention and one or more excipients, such compositions are preferably semi-solid. It further provides methods of manufacturing such compositions, uses of such compositions and specific forms of the agent of the invention. Particularly, the invention provides in a first aspect a topical pharmaceutical composition of the solution type comprising the agent of the invention; in a second aspect a topical pharmaceutical composition of the suspension type comprising the agent of the invention; in a third aspect a process for producing 6-(6-hydroxymethyl-pyrimidin-4-yloxy)-naphthalene-1-carboxylic acid (3-trifluoromethyl-phenyl)-amide or a salt, or a polymorph, or a solvate thereof; in a fourth aspect methods for manufacturing compositions comprising 6-(6-hydroxymethyl-pyrimidin-4-yloxy)-naphthalene-1-carboxylic acid (3-trifluoromethyl-phenyl)-amide or a salt, or a polymorph, or a solvate thereof; in a fifth aspect the use of such compositions as pharmaceutical, particularly as pharmaceutical for the treatment of dermatological diseases, the use of 6-(6-hydroxymethyl-pyrimidin-4-yloxy)-naphthalene-1-carboxylic acid (3-trifluoromethyl-phenyl)-amide or a salt, or a polymorph, or a solvate thereof as pharmaceutical for the treatment of dermatological diseases and in a sixth aspect specific forms of the agent of the invention, methods of using and methods of manufacturing such specific forms.

### BRIEF DESCRIPTION OF THE DRAWINGS

FIG. 1 discloses the XRPD pattern of Form B recorded by reflexion mode.

FIG. 2 discloses the XRPD pattern of Form B (highly crystalline material) recorded by reflexion mode

FIG. 3 discloses the XRPD pattern of Form A recorded by reflexion mode

FIG. 4 discloses the XRPD pattern of Form B recorded by transmission mode

FIG. 5 discloses the XRPD pattern of Form A recorded by transmission mode

FIG. 6 discloses the microscopic observation of Variant E, showing crystal of the agent of the invention

FIG. 7 discloses the microscopic observation of Variant E, showing cetyl/stearyl crystals

FIG. 8 discloses the microscopic observation of Variant C

FIG. 9 discloses the macroscopic observation of Variant B

### DETAILED DESCRIPTION OF PREFERRED EMBODIMENTS

The invention may be more fully appreciated and objects other than those set forth above will become apparent when consideration is given to the following description, including the following glossary of terms and the concluding examples.

As used herein, the terms “including”, “containing” and “comprising” are used herein in their open, non-limiting sense. Where the plural form (e.g. compounds, excipients) is used, this includes the singular (e.g. a single compound, a single excipient). “A compound” does not exclude that (e.g. in a pharmaceutical composition) more than one compound (or a salt thereof) is present.

The agent of the invention, 6-(6-hydroxymethyl-pyrimidin-4-yloxy)-naphthalene-1-carboxylic acid (3-trifluoromethyl-phenyl)-amide, is intended to represent amorphous and crystalline forms such as polymorphs. The agent of the invention is intended to also represent a solvate thereof, particularly a hemihydrate, a pharmaceutical acceptable salt thereof and its mixtures. The agent of the invention is intended to also represent material exhibiting specific solid state properties such as milled forms.

It is further understood that the various embodiments, preferences and ranges of this invention, as provided/disclosed in the specification and claims may be combined with other specified features to provide further embodiments.

Further, depending on the specific embodiment, selected definitions, embodiments or ranges may not apply. The following general definitions shall apply in this specification, unless otherwise specified:

As used herein, the term “Solvate” refers to a crystal form of a compound which additionally contains one or more types of solvent molecules in a stoichiometrically defined amount. Preferably, solvates contain one type of solvent molecule, such as water, in the crystal lattice.

It is further understood that the agent of the invention in various embodiments, may be intended to comprise a prodrug thereof.

As used herein, the term “Prodrug” indicates a compound that may be converted under physiological conditions or by solvolysis to a biologically active compound of the invention. Thus, this term refers to a metabolic precursor of an agent of the invention that is pharmaceutical acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism. Prodrugs of a agent of the invention may be prepared by modifying functional groups present in the agent of the invention in such a way that the modifications are cleaved, either in routine manipulation or in vivo, to the parent compound of the invention. Prodrugs include compounds of the invention wherein a hydroxyl group is bonded to any group that, when the prodrug of the agent of the invention is administered to a mammalian subject, cleaves to form a free hydroxy group. Examples of prodrugs include, but are not limited to, acetate, formate and benzoate derivatives of alcohol groups in the agent of the invention. Suitable prodrugs include pharmaceutically acceptable esters of the agent of the invention. As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms, particularly formates, acetates, propionates, butyrates, acrylates and ethylsuccinates.

As used herein, the term “pharmaceutically acceptable salts” refers to the nontoxic acid or alkaline earth metal salts of the compounds of the invention. These salts can be prepared in situ during the final isolation and purification of the compounds, or by separately reacting the base or acid functions with a suitable organic or inorganic acid or base, respectively. Representative salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydro-chloride, hydrobromide, hydroiodide, 2-hydroxyethanesulfonate, lactate, maleate, methane-sulfonate, nicotinate, 2-naphth-alenesulfonate, oxalate, pamoate, pectinate, persulfate, 3-phenylproionate, picrate, pivalate, propionate, succinate, sulfate, tartrate, thiocyanate, p-toluene-sulfonate, and undecanoate. Also, basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others. Water or oil-soluble or dispersible products are thereby obtained. Basic addition salts can be prepared in situ during the final isolation and purification of the compounds, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethyl-amine, trimethylamine, triethylamine, ethylamine, and the like. Other representative organic amines useful for the formation of base addition salts include diethylamine, ethylenediamine, ethanolamine, diethanolamine, piperazine, pyridine, picoline, triethanolamine and the like and basic amino acids such as arginine, lysine and ornithine.

As used herein, the term “penetration enhancer” refers to a substance that enhances, i.e. improves, the penetration of the agent of the invention when administered topically, (epicutanously), into skin or mucosa, e.g. into skin, such as the lower epidermis and the dermis, compared with the penetration for the agent of the invention without that penetration enhancer. A penetration enhancer as used herein is added in an effective amount, meaning in amount of at least 2.5 wt-%. This enhanced penetration will lead to higher levels within the skin, in particular in the lower epidermis and the dermis. Higher penetration may also result in an increased permeation, e.g. increased permeation through the skin. Preferably, the delivery of the agent of the invention to the systemic circulation is not or not significantly enhanced (no or no significant permeation).

As used herein, the term “topical pharmaceutical composition” is known in the field (e.g. see European Pharmacopoeia, 6.3, 01/2009, 0132) and particularly refers to a composition of the solution type or the suspension type. Such compositions contain (i.e. comprise or consist of) i) the agent of the invention and ii) a matrix. The matrix (also referred to as “base”) contains pharmaceutically acceptable excipients and is adapted to a topical application. Further, compositions of the invention may be formulated as semi-solid, patch, gel, foam, tincture, solution, (lip) stick, or spray; each of them either in the suspension type or the solution type. Consequently, viscosities of the compositions of the invention, both solution type and suspension type, may vary over a broad range, typically they are semi-solid or liquid, preferably semi-solid. Compositions of the solution type are characterized in that the agent of the invention is dissolved in the matrix; preferably in the form of a “hydrophilic ointment”. Compositions of the suspension type are characterized in that the agent of the invention is suspended in the matrix; preferably in the form of a “hydrophobic ointment”.

“XRPD” means X ray powder diffraction.

WO 2006/059234 suggests oral administration of certain naphthalene-1-carboxylic acid derivatives, such as 6-(6-hydroxymethyl-pyrimidin-4-yloxy)-naphthalene-1-carboxylic acid (3-trifluoromethyl-phenyl)-amide and very generally discloses pharmaceutical compositions in unit dosage form, such as capsules.

Patients suffering from skin diseases may profit from topical treatment with a VEGF inhibitor. Hence, it is an object of the invention to provide topical pharmaceutical compositions comprising 6-(6-hydroxymethyl-pyrimidin-4-yloxy)-naphthalene-1-carboxylic acid (3-trifluoromethyl-phenyl)-amide with desirable properties such as efficacy, good bioavailability, good skin penetration, low potential for skin irritation, good stability, low risk for provoking allergic reactions, reasonable absorption time and favorable cosmetic parameters such as smell, fluidity, spreadability, skin sensation and potential to produce a film residue.

Thus, in a first aspect, the invention relates to a topical pharmaceutical composition containing (i.e. comprising or consisting of) i) the agent of the invention or a solvate thereof and ii) a hydrophilic matrix. Such composition is typically of the solution type.

It was found by the present inventors that such compositions provide enhanced skin penetration. This was surprising; especially in view of the fact that the agent of the invention has very poor solubility properties in both hydrophilic and hydrophobic media (i.e. low solubility in aqueous and oily media). By the use of hydrophilic matrix as defined below it was possible to increase the level of the agent of the invention to a pharmaceutically beneficial level without skin irritation. Further, these compositions show good physical and chemical stability. This aspect of the invention shall be explained in further detail below:

Agent of the invention: The agent of the invention is a known compound and may be obtained according to the methods described herein. Particularly suitable for the inventive compositions are agents of the invention in crystalline form as described herein. The amount of agent of the invention in the inventive composition may vary over a broad range, it is typically provided in an effective amount. An effective amount refers to an amount of the agent of the invention which, when administered to a mammal, particularly a human, is sufficient to effect a treatment as defined below. Suitable amounts for the agent of the invention may be determined by the skilled person in routine experiments; typically they are in the range between 0.2-5 wt-%, preferably 0.5-2.0 wt-%, such as 0.5, 0.8 or 1.0 wt-% of the total composition.

Hydrophilic matrix: According to this aspect of the invention, the hydrophilic matrix contains one or more types of polyethylene glycol (PEG) and optionally water; preferably at least two types of PEG and water. It was found that such matrix dissolves a high amount of agent of the invention and reduces skin dehydration. PEGs are polyadducts of ethylene oxide and are defined by their molecular mass (which is indicated as number behind the abbreviation PEG). Suitable are PEGs with molecular masses in the range of 100-25000 g/mol, particularly 400-10000 g/mol. The term “one or more types of PEG” refers to either the use of a PEG having one molecular mass in the inventive composition (e.g. PEG 400 as the only type of PEG present in the composition) or the use of two or more PEGs having different molecular masses (e.g. PEG 400+PEG 3000 or PEG 400 and PEG 4000 being present in the composition). Advantageously, the hydrophilic matrix contains low molecular weight PEG (e.g. 200-1000 g/mol) and high molecular weight PEG (e.g. 2000-5000 g/mol). Preferably, the hydrophilic matrix contains low molecular weight PEG (e.g. 400 g/mol) and high molecular weight PEG (e.g. 4000 g/mol). PEGs are known excipients for pharmaceutical compositions and are commercially available. The amounts of water and PEG depend on the intended type of composition (cream, spray . . . ) and may be readily adapted be the skilled person. A suitable hydrophilic matrix may contain up to 40 wt. % water, preferably 10-20 wt. % water. A suitable hydrophilic matrix may contain at least 50 wt. % PEG, preferably 75-95 wt. % PEG. Further, a suitable hydrophilic matrix may contain between 10-80 wt. % low molecular PEG and between 10-80 wt. % high molecular PEG. Further, a suitable hydrophilic matrix may contain low molecular weight PEG and high molecular weight PEG in a ratio between 4:1 to 1:1, preferably 2.5:1 to 1.5:1.

In one embodiment, the invention relates to a composition according to this aspect of the invention which contains no further excipients. Thus, the composition only contains an agent of the invention, one or more PEGs and optionally water, preferably an agent of the invention, two or more PEGs and water. Such compositions are considered advantageous e.g. for simple manufacturing and/or for patient populations with increased skin irritation/allergic potential towards other excipients.

In a further embodiment, the invention relates to a composition according to this aspect of the invention which contains an agent of the invention, one or more PEGs, optionally water, optionally one or more excipients as defined below but which does not contain an effective amount of a penetration enhancer, meaning a penetration enhancer in amounts of at least 2.5 wt-% The present inventors found that a composition as described in this first aspect of the invention does not require a penetration enhancer to achieve a therapeutic effect. This is surprising, as the prior art suggest a beneficial effect of oleyl alcohol as penetration for compounds with related chemical structure. Compositions without an effective amount of a penetration enhancer are considered advantageous e.g. for simple manufacturing and/or for patient populations with increased skin irritation/allergic potential.

In a further embodiment, the invention relates to a composition according to this aspect of the invention which contains one or more additional excipients. Such excipients are known in the field and may be readily identified by the skilled person. Suitable excipients may be selected from the group consisting of antioxidants, gelling agents, ph adjusting agents/buffers, agents to modify consistency, preservatives, (co-)solvents, fillers, binders, disintegrators, flow conditioners, lubricants, fragrances, stabilizers, wetting agents, emulsifiers, solubilizers and salts for regulating osmotic pressure. Such excipients are known in the field, commercially available and may be identified in standard textbooks, such as the Handbook of Pharmaceutical Excipients by R. C. Rowe et al. Such compositions are advantageous to specifically adapt to manufacturers or patients needs and thus improve product properties (like shelf life or patient compliance). Suitable further excipients are explained below:

Antioxidants are known in the field and may be selected by a skilled person to be compatible with the final pharmaceutical composition. It is understood that one or more antioxidants may be used. It was found that the antioxidant stabilizes the agent of the invention. Preferably, the antioxidant is selected from the group consisting of phenole derivatives (e.g. butylated hydroxytoluene (BHT), butylated hydroxyanisole (BHA)); ascorbic acid derivatives (e.g. ascorbic acid, ascorbyl palmiate), tocopherol derivatives (e.g. Vitamin E, Vitamin E TPGS), bisulfite derivatives (Na bisulfite, Na meta bisulfite) and thio urea. More preferably, is selected from the group consisting of butylated hydroxytoluene (BHT), butylated hydroxyanisole (BHA), alpha tocopherol, ascorbic acid or a mixture of thereof. Particularly preferably, the antioxidant is BHT. A suitable composition may contain up to 2 wt % antioxidant, preferably 0.005-0.5 wt %.

Gelling agents are known in the field and may be selected by a skilled person to be compatible with the final pharmaceutical composition. It is understood that one or more gelling agents may be used. Gelling agents are included in the compositions of this invention to adjust viscosity. Preferably, gelling agents are acrylic acid derivatives or cellulose derivatives, such as hydroxypropylcellulose. A suitable composition may contain up to 10 wt % gelling agent, preferably 0.02 to 2 wt %.

Agents to adjust the pH or to provide a pH buffer are known in the field. Appropriate acids or bases may be selected by a skilled person to be compatible with the final pharmaceutical composition. It is understood that one or more of such agents may be used, such as citric acid. A suitable composition may contain such acids/bases to adjust the pH of the inventive composition in the range of 4-8, preferably 5-7, such as 6.5.

Agents to modify consistency, also named consistency improver, are known in the field. Appropriate compounds may be selected by a skilled person to be compatible with the final pharmaceutical composition. It is understood that one or more of such agents may be used, e.g. cetyl alcohol, stearyl alcohol and mixtures thereof. A suitable composition may contain 0.1 to 2 wt %.

Preservatives are known in the field and may be selected by a skilled person to be compatible with the final pharmaceutical composition. It is understood that one or more preservatives may be used. Preservatives are included in the pharmaceutical compositions of this invention to increase shelf life. Preferably, preservatives are selected from the group of acids (e.g. sorbic acid, benzoic acid); alcohols (e.g. benzyl alcohol), quaternary amines, phenols, and parahydroxybenzoates. More preferably, preservatives are selected from parabens, alcohols, quaternary ammoniums, biguanides, mercuric salts, imidurea, acids, such as benzoic acid. Particular preferably, the preservative is benzyl alcohol. Also particular preferably, the preservative is benzoic acid. A suitable composition may contain up to 5 wt %, preferably 0.01 to 3 wt %.

Co-solvents and solvents are known in the field and may be selected by a skilled person to be compatible with the final pharmaceutical composition; it denotes an excipient which dissolves the agent of the invention (partly or fully) and has a high miscibility with water. A solvent is an excipient which dissolves the agent of the invention but has a low miscibility with water. Thus, depending on the type of composition and the other excipients present, a specific compound my serve as a solvent or as a co-solvent. It is understood that one or more co-solvents/solvents may be used.

The invention relates in a second aspect to a topical pharmaceutical composition containing i) the agent of the invention or a solvate thereof; ii) a hydrophobic matrix; and iii) a penetration enhancer. Such composition is typically of the suspension type.

It was found by the present inventors that such compositions provide significantly enhanced skin penetration. This was surprising; especially in view of the fact that the agent of the invention is suspended in the matrix and thus only a small fraction of molecules is dissolved and available for penetration. By the use of a penetration enhancer it was possible to increase the level of the agent of the invention to a pharmaceutically beneficial level without skin irritation. Further, these compositions show good physical and chemical stability. This aspect of the invention shall be explained in further detail below:

Agent of the invention: The agent of the invention is a known compound and may be obtained according to the methods described herein. Particularly suitable for the inventive compositions are agents of the invention in crystalline form as described herein. The amount of agent of the invention in the inventive composition may vary over a broad range, it is typically provided in an effective amount. An effective amount refers to an amount of the agent of the invention which, when administered to a mammal (preferably a human), is sufficient to effect a treatment as defined below. Suitable amounts for the agent of the invention may be determined by the skilled person in routine experiments; typically they are in the range between 0.2-5 wt-%, preferably 0.5-2.0 wt-%, such as 0.5, 0.8 or 1.0 wt. %.

Hydrophobic matrix: According to this aspect of the invention, the matrix contains paraffines (hard, liquid, light liquid), vegetable oils, animal fats, synthetic glycerides, waxes and/or liquid polysiloxanes. Typically, the hydrophobic matrix can absorb only small amounts of water. Preferably, the hydrophobic matrix contains one or more types of hydrocarbons; preferably at least two types of hydrocarbons. It was found that such matrix disperses a high amount of agent of the invention and produces a stable composition. Suitable hydrocarbons are known in the field and may be selected by a skilled person to be compatible with the final pharmaceutical composition. Suitable hydrocarbons include solid and liquid hydrocarbons which may be linear and/or branched. Such hydrocarbons are known excipients for pharmaceutical compositions and are commercially available (e.g. as mixtures of individual components). Suitable hydrocarbons include “mineral oil”, “petrolatum”, “microcrystalline wax”. A suitable hydrophobic matrix may contain up to 66 wt. % mineral oil, preferably 20-40 wt. % mineral oil. A suitable hydrophobic matrix may contain up to 98 wt. % petrolatum, preferably 40-60 wt. % petrolatum. A suitable hydrophobic matrix may contain up to 25 wt. % microcrystalline wax, preferably 5-20 wt. % microcrystalline wax. A suitable hydrophobic matrix may contain mineral oil and petrolatum in a ratio between 1:1 to 1:3, preferably 1:1.5 to 1:2.0. Further, a suitable hydrophobic matrix may contain mineral oil and microcrystalline wax in a ratio between 1:0.2 to 1:1, preferably 1:0.33 to 1:0.66.

Penetration enhancer: The penetration enhancer is defined above; a wide range of penetration enhancers may be used. Particularly suitable are penetration enhancers selected from the group consisting of saturated fatty acids and saturated fatty acid esters. Preferred are saturated C6-C30 fatty acids, -esters; particularly preferred are C10-C20 fatty acids, -esters. Further, linear fatty acids, -esters are preferred. For esters, C1-C4 alkyl groups are preferred. Among these penetration enhancers, isopropyl myristate is particularly suitable. The amount of penetration enhancer in the inventive composition may vary over a broad range, it is typically provided in an effective amount. Suitable amounts of penetration enhancer may be determined by the skilled person in routine experiments; typically they are between 2.5-20 wt-%, preferably 2.5-10 wt-% of the total composition.

In one embodiment, the invention relates to a composition according to this aspect of the invention which contains no further excipients. Thus, the inventive composition only contains (i.e. consist of or essentially consists of) an agent of the invention, one or more hydrocarbons and a penetration enhancer. Such compositions are considered advantageous e.g. for simple manufacturing and/or for patient populations with increased skin irritation/allergic potential towards other excipients.

The invention relates in a third aspect to a new process to produce 6-(6-hydroxymethyl-pyrimidin-4-yloxy)-naphthalene-1-carboxylic acid (3-trifluoromethyl-phenyl)-amide or a salt, or a polymorph, or a solvate thereof.

Desirable properties of a process suitable to produce pharmaceutical compounds and/or a pharmaceutical agent or a salt or solvate are for example efficiency, low number of steps, high yield, low costs of goods, high safety profile, selectivity and fast reaction times.

A process for preparing naphthalene-1-carboxylic acid derivatives, such as 6-(6-hydroxymethyl-pyrimidin-4-yloxy)-naphthalene-1-carboxylic acid (3-trifluoromethyl-phenyl)-amide is known. WO 2006/059234 discloses the preparation of 6-(6-hydroxymethyl-pyrimidin-4-yloxy)-naphthalene-1-carboxylic acid (3-trifluoromethyl-phenyl)-amide. In the preparation of said compound, 6-hydroxy-1-naphthoic acid is coupled with 4,6-dichloro-pyrimidine, the resulting 6-(6-chloro-pyrimidin-4-yloxy)-naphthalene-1-carboxylic acid is, through amide coupling conditions with 3-trifluormethyl-aniline, transformed into 6-(6-chloro-pyrimidin-4-yloxy)-naphthalene-1-carboxylic acid (3-trifluoromethyl-phenyl)-amide. 6-(6-Chloro-pyrimidin-4-yloxy)-naphthalene-1-carboxylic acid (3-trifluoromethyl-phenyl)-amide is then converted to 6-[5-(3-trifluoromethyl-phenylcarbamoyl)-naphthalen-2-yloxy]-pyrimidine-4-carboxylic acid ethyl ester through catalytic carboxylation conditions. Subsequently 6-[5-(3-trifluoromethyl-phenylcarbamoyl)-naphthalen-2-yloxy]-pyrimidine-4-carboxylic acid ethyl ester is reduced to give 6-(6-hydroxymethyl-pyrimidin-4-yloxy)-naphthalene-1-carboxylic acid (3-trifluoromethyl-phenyl)-amide.

Major drawbacks of said process are that the carboxylation step requires very high pressure together with high temperature. Special equipment is required to mitigate the risk with this high pressure and high temperature reaction. A high loading of the Palladium catalyst is required for the carboxylation step and the reaction is proceeding with slow conversion. Since the carboxylation step is at late stage in the process, a risk of heavy metal contamination of 6-(6-hydroxymethyl-pyrimidin-4-yloxy)-naphthalene-1-carboxylic acid (3-trifluoromethyl-phenyl)-amide is present.

The reduction step is low yielding, leading to the formation of 6-hydroxy-naphthalene-1-carboxylic acid (3-trifluoromethyl-phenyl)-amide as a main side product and requires a laborious separation step in order to purify the 6-(6-hydroxymethyl-pyrimidin-4-yloxy)-naphthalene-1-carboxylic acid (3-trifluoromethyl-phenyl)-amide.

The process introduces a functional group in the wrong oxidation stage requiring oxidation state adjustments and is not suitable for the synthesis of larger quantities of 6-(6-hydroxymethyl-pyrimidin-4-yloxy)-naphthalene-1-carboxylic acid (3-trifluoromethyl-phenyl)-amide.

It is hence an object of the present invention to provide an alternative process for preparing 6-(6-hydroxymethyl-pyrimidin-4-yloxy)-naphthalene-1-carboxylic acid (3-trifluoromethyl-phenyl)-amide, or a salt or solvate thereof, preferably a reaction route which avoids the above-mentioned drawbacks of the prior art process.

The new processes, according to the present invention, for producing the hemihydrate of 6-(6-hydroxymethyl-pyrimidin-4-yloxy)-naphthalene-1-carboxylic acid (3-trifluoromethyl-phenyl)-amide in milled form (14′), involving Sections A, B, C, D and E or Sections B, C, D and E or Sections C, D and E; for producing the hemihydrate of 6-(6-hydroxymethyl-pyrimidin-4-yloxy)-naphthalene-1-carboxylic acid (3-trifluoromethyl-phenyl)-amide (14) involving Sections A, B, C and D or Sections B, C and D or Sections C and D; for producing 6-(6-hydroxymethyl-pyrimidin-4-yloxy)-naphthalene-1-carboxylic acid (3-trifluoromethyl-phenyl)-amide in free form or as a salt thereof, as defined herein (13) involving Sections A, B and C or Sections B and C; or salt thereof, as defined herein, are summarized in Scheme 1. Under certain conditions the hemihydrate of 6-(6-hydroxymethyl-pyrimidin-4-yloxy)-naphthalene-1-carboxylic acid (3-trifluoromethyl-phenyl)-amide (14) can be produced involving Sections A, B and C′ or Sections B and C′ and the hemihydrate of 6-(6-hydroxymethyl-pyrimidin-4-yloxy)-naphthalene-1-carboxylic acid (3-trifluoromethyl-phenyl)-amide in milled form (14′) can be produced involving Sections A, B, C′ and E or Sections B, C′ and E or Sections C′ and E.

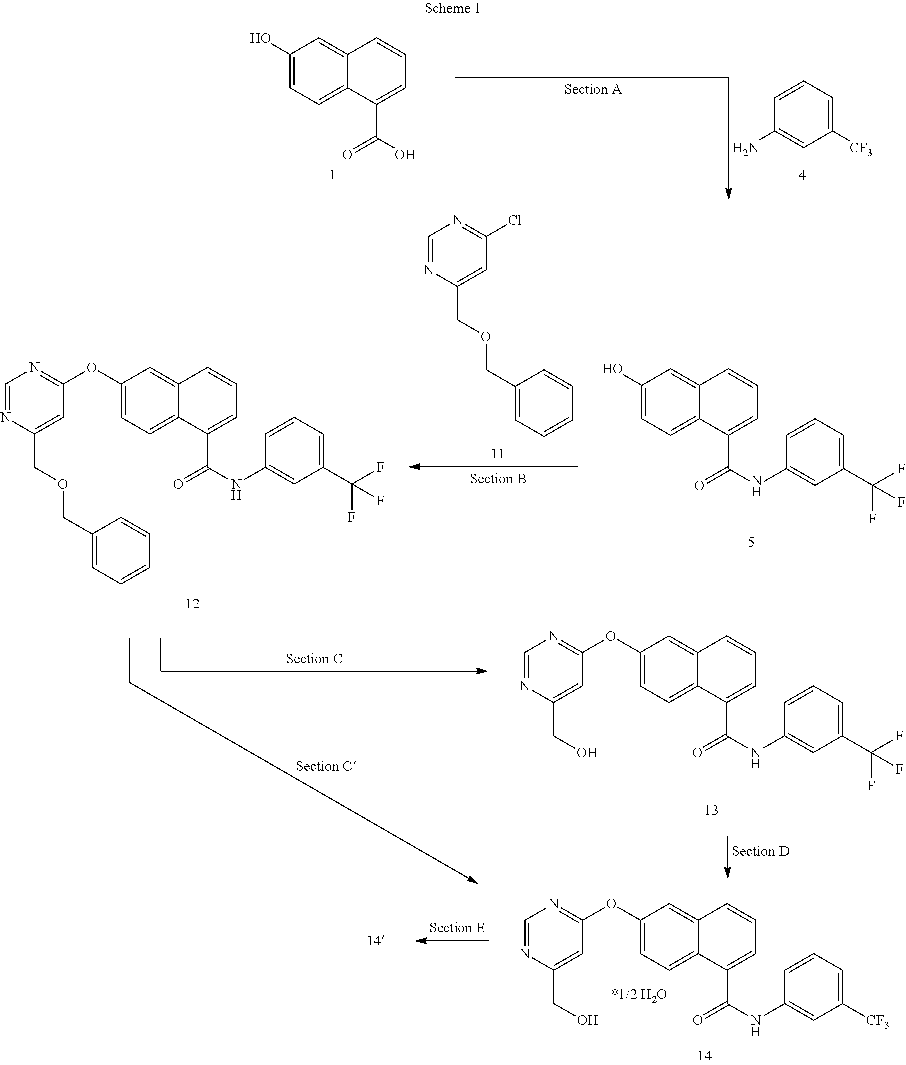

Namely, a compound of formula (1) is coupled with a compound of formula (4) resulting in a compound of formula (5), or a salt thereof, according to a method described in Section A.

The compound of formula (5), or a salt thereof is then converted into a compound of formula (12), or salt thereof, according to a method described in Section B. The compound of formula (12), or a salt thereof is then converted into a compound of formula (13), or salt thereof, according to a method described in Section C. The compound of formula (13), or a salt thereof is then optionally converted into a hemihydrate of formula (14), according to a method described in Section D. The hemihydrate of formula (14) is then optionally milled and/or delumped to lead to a milled form (14′) of a hemihydrate of formula (14), according to a method described in Section E. Alternatively, the compound of formula (12), or a salt thereof is converted into a hemihydrate of formula (14) according to a method described in Section C′.

As discussed below, Sections A, B, C, C′ and D as such are also preferred embodiments of the present invention.

### Section A: Preparation of a Compound of Formula (4)

In one embodiment, the invention relates to a process for preparing a compound of formula (5), or salt thereof,

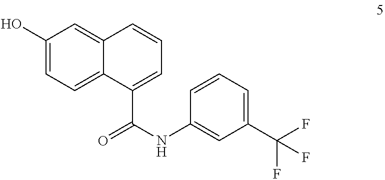

said process comprising reacting a compound of formula (1), or salt thereof,

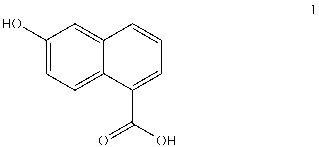

with the aniline of formula (4) or salt thereof,

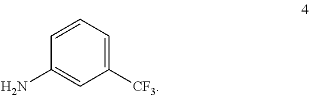

The processes, according to the present invention, to react a compound of formula (1), as defined herein, with a compound of formula (4), as defined herein, to form a compound of formula (5), as defined herein, are outlined in Scheme 2.

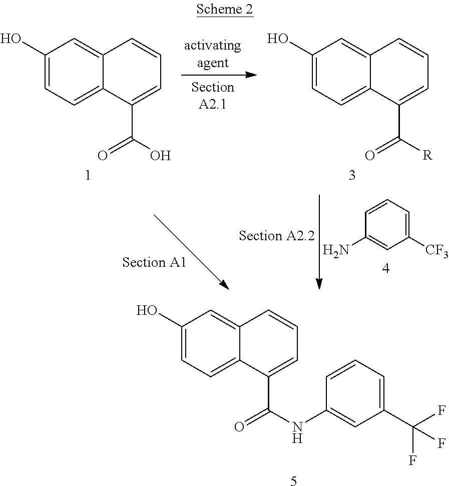

The reaction to obtain the amide of formula (5) from the acid of formula (1) and the aniline of formula (4) can take place neat or in a suitable inert solvent, preferably in an aprotic solvent such as esters, for example ethyl acetate, or isopyl acetate; N-methyl-2-pyrrolidinone; acetonitrile; halogenated hydrocarbons, for example methylene chloride; ethers, for example THF, 2-methyltetrahydrofuran, dimethoxyethane, or dioxane; or aromatic solvents for example benzene, chlorobenzene, toluene, phenylethane or xylenes; or mixtures thereof; in the presence of an activating agent such as propane phosphonic anhydride; thionyl chloride; oxalyl chloride; 4-(4,6-dimethoxy-1,3,5-triazin-2-yl)-4-methylmorpholinium chloride (DMTMM), or suitable carbodiimides like for example di-cyclohexyl-carbodiimide (DCC), N,N′-Diisopropylcarbodiimide (DIC), 1-Ethyl-3-(3-dimethylaminopropyl)carbodimide (EDC). These activating agents can be purchased from suppliers, such as Aldrich, Fluka or Acros. The reaction can be either performed stepwise, first activating the compound of formula (1) by reaction with an activating agent (Section A2.1), with isolation of the activated intermediate of formula (3), wherein R is an activating group, then coupling of the activated intermediate of formula (3) with the aniline of formula (4) (Section A2.2), or as a one step procedure (Section A1). If a stepwise procedure is used, a solvent change may be involved. Typically, the reaction can be conducted at 0° C. to reflux, preferably 0 to 200° C., more preferably 0 to 150° C., yet more preferably 10 to 80° C., most preferably 60 to 90° C.

Preferably, when DMTMM is used as activating agent, a stepwise procedure is used. The activating step than preferably takes place in acetonitrile at a temperature of 10 to 20° C. and the coupling step preferably takes place in N-methyl-2-pyrrolidinone at a temperature of 20 to 55° C.

Preferably, when thionyl chloride; oxalyl chloride, are used as activating agent, a one step procedure is used.

### Section B: Preparation of a Compound of Formula (12)

In another embodiment, the present invention relates to a process for preparing a compound of formula (12), or a salt thereof,

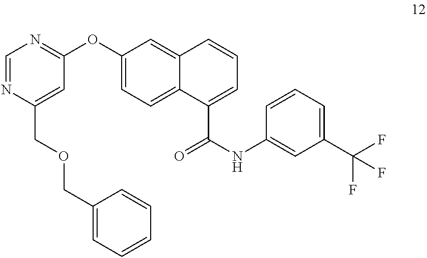

said process comprising reacting a compound of formula (5), or salt thereof,

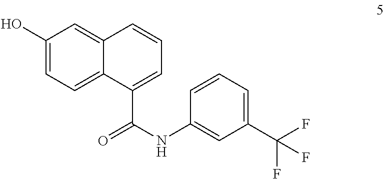

with the compound of formula (11) or salt thereof,

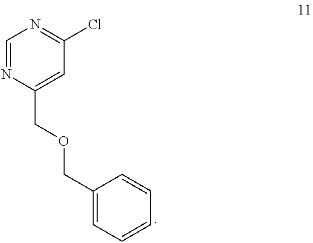

The reaction to obtain the benzyl ether of formula (12) from the coupling of a compound of formula (5) with a compound of formula (11) can take place in a suitable inert solvent, preferably in an aprotic, polar solvent such as N-methyl-2-pyrrolidinone (NMP); dimethylformamide (DMF); dimethylsulfoxide (DMSO); ethers for example tetrahydrofurane, 2-methyltetrafurane, tert-butyl methyl ether; or esters for example ethyl acetate or isopropyl acetate; or acetonitril; or in a solvent such as halogenated hydrocarbons, for example methylene chloride; in the presence of a base, for example potassium carbonate or cesium carbonate. Typically, the reaction can be conducted at 20° C. to reflux, preferably 20 to 200° C., more preferably 40 to 150° C., most preferably 80 to 100° C. Preferably, potassium carbonate is used as base and the reaction preferably takes place in N-methyl-2-pyrrolidinone at a temperature of 100° C.

The compound of formula (11) shows an exothermic degradation reaction beginning at approx. 80-90° C. with a release of approx. −990 kJ/kg.

It has been surprisingly found that the reaction can be safely conducted by adding a cold solution of the compound of formula (11) to a heated mixture of the compound of formula (5) and the base in a suitable solvent at the reaction temperature, whereby the addition takes place at the approximate speed of consumption of the compound of formula (11).

### Section C: Preparation of a Compound of Formula (13)

In another embodiment, the present invention relates to a process for preparing a compound of formula (13), or a salt thereof,

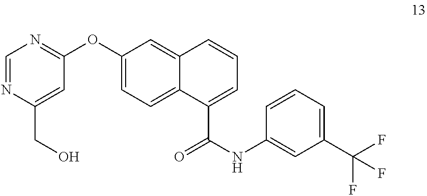

from a compound of formula (12), or salt thereof,

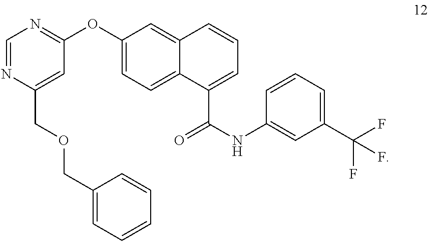

The processes, according to the present invention, to convert a compound of formula (12), as defined herein to a compound of formula (13), as defined herein, are outlined in Scheme 3.

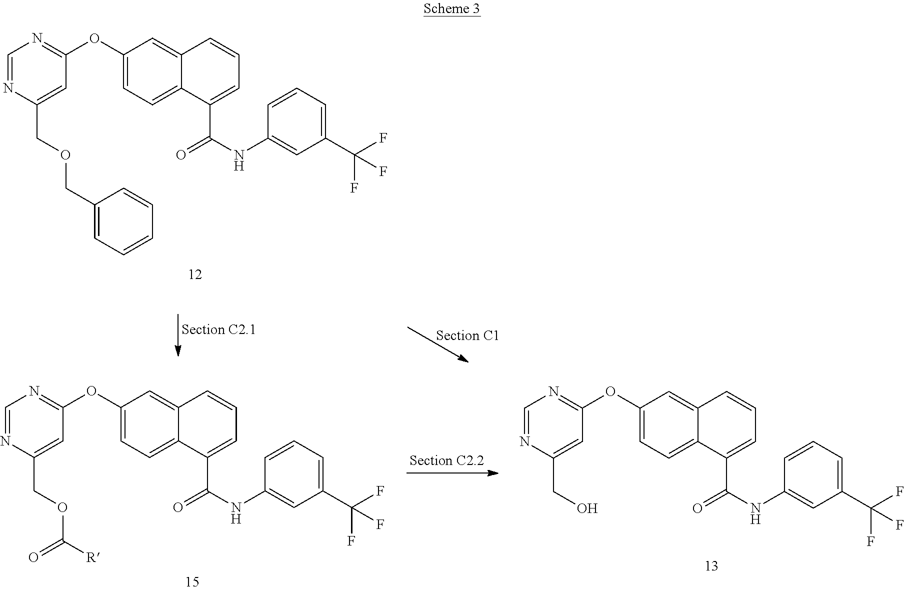

### Section C1 One Step Procedure

The reaction to obtain the alcohol of formula (13) from the benzyl ether of formula (12) can take place neat or in inert organic solvents, such as halogenated hydrocarbons, such as methylene chloride; alcohols, such as ethanol, methanol, 2-propanol, 1-propanol or ethers, such as tetrahydrofuran, 2-methyltetrahydrofurane, dimethoxyethane, tert-butyl methyl ether, or dioxane; or esters for example ethyl acetate or isopropyl acetate, or acetonitril or aromatic solvents such as chlorobenzene, toluene, cumene, anisol or xylenes or mixtures thereof in the presence of strong acids like methanesulfonic acid, trifluoroacetic acid. Typically, the reaction can be conducted at −15° C. to reflux, preferably −10 to 150° C., most preferably −5 to 100° C. Preferably, of trifluoroacetic acid (25 eq) in toluene at 100° C. or methanesulfonic acid (20 eq) in dichloromethane at −5-20° C. are used for the conversion.

Suitable conditions for the conversion of the compound of formula (12) into the compound of formula (13), using sulfuric acid, hydrochloric acid or hydrobromic acid, were not found despite several conditions being tested.

Surprisingly, the use of trifluoroacetic acid or methanesulfonic acid gave the compound of formula (13) in high yield with clean conversion.

### Sections C2.1, C2.2 Two Step Procedure

Alternatively, the alcohol of formula (13) can be prepared via acylation (Section C2.1) of a compound of formula (12) to form a compound of formula (15), wherein R′ is selected from C₁-C₇-alkyl, followed by deprotection of a compound of formula (15) with a suitable base (Section 2.2). The acylation step (Section C2.1) can take place neat or in a suitable inert solvent, preferably in an aprotic solvent such as halogenated hydrocarbons, for example methylene chloride; ethers, for example THF, 2-methyltetrahydrofurane, dimethoxyethane, or dioxane; or aromatic solvents for example benzene, chlorobenzene, toluene, phenylethane or xylenes or mixtures thereof; in the presence of an activating agent such as acyl chlorides or acid anhydrides and optionally in the presence of an inorganic acid for example sulfuric acid or hydrochloric acid. Typically, the reaction can be conducted at 0° C. to reflux, preferably 0 to 200° C., more preferably 0 to 150° C., yet more preferably 10 to 80° C., most preferably 40 to 70° C. Preferably, the reaction is performed neat using acetic anhydride as activating agent and an acid, preferably sulfuric acid is added to the mixture.

The compound of formula (15) can optionally be isolated and purified.

The deprotection step preferably takes place neat or in a suitable inert solvent, preferably in an aprotic solvent such as halogenated hydrocarbons, for example methylene chloride; ethers, for example THF, 2-methyltetrahydrofurane, dimethoxyethane, or dioxane; or aromatic solvents for example benzene, chlorobenzene, toluene, phenylethane or xylenes, or in a protic solvent such as alcohols, for example ethanol, methanol, 2-propanol, 1-propanol or mixtures thereof in the presence of a suitable inorganic base for example sodium alkoxides, potassium alkoxides, sodium hydroxide, potassium hydroxide, sodium carbonate or potassium carbonate. Typically, the reaction can be conducted at 0° C. to reflux, preferably 0 to 200° C., more preferably 0 to 150° C., yet more preferably 10 to 80° C., most preferably 40 to 70° C. Preferably, the reaction is performed in a mixture of methanol and 2-methyltetrahydrofurane in the presence of sodium methoxide.

Suitable hydrogenation conditions for the conversion of the compound of formula (12) into the compound of formula (13) were not found, despite several conditions being tested.

The new process proved however surprisingly beneficial for the conversion of the compound of formula (12) into the compound of formula (13). Preferably the two step procedure via Section C2.1 and C2.2 is used.

### Section C′: Preparation of a Compound of Formula (14)

In another embodiment, the present invention relates to a process for preparing a hemihydrate of formula (14),

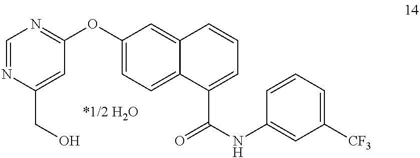

from a compound of formula (12), or salt thereof,

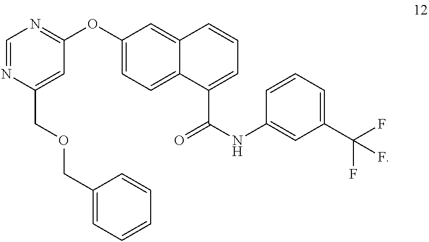

Suitable conditions for the conversion are mentioned below in Section C. It was found that, involving water during the work-up of procedure followed by crystallizing, the hemihydrate of formula (14) can be obtained directly from the conversion step. Suitable conditions for the crystallization are mentioned below in the section relating to the sixth aspect of the invention, namely specific forms of the agent of the invention.

### Section D: Preparation of a Hemihydrate of Formula (14)

In another embodiment, the present invention relates to a process for preparing a hemihydrate of formula (14),

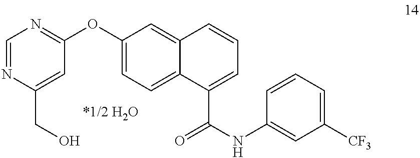

said process comprises crystallizing a compound of formula (13), or salt thereof,

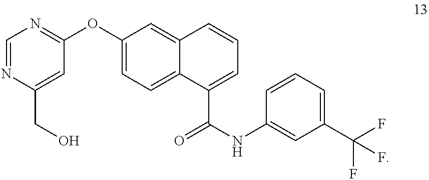

Suitable conditions for the crystallization are mentioned below in the section relating to the sixth aspect of the invention, namely specific forms of the agent of the invention.

### Section E: Preparation of Milled Hemihydrate (14)

In another embodiment, the present invention relates to a process for preparing milled hemihydrate (14) by milling and/or delumping a hemihydrate of formula (14).

Another preferred embodiment of the invention is a process comprising sections B, C, optionally D and optionally E.

Another preferred embodiment of the invention is a process comprising sections B, C′ and optionally E.

Another preferred embodiment of the invention is a process comprising sections C, optionally D and optionally E.

Another preferred embodiment of the invention is a process comprising sections C′ and optionally E.

In another embodiment, the present invention relates to an intermediate of formula (12), or salt thereof,

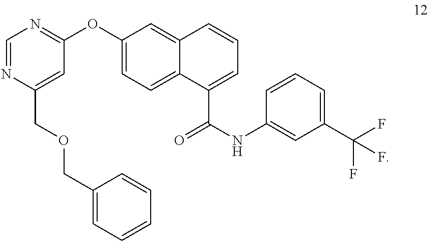

In another embodiment, the present invention relates to an intermediate of formula (15), or salt thereof,

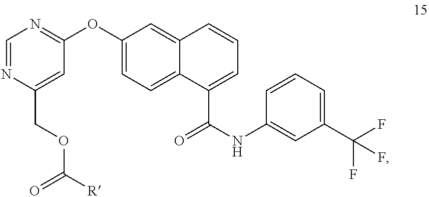

wherein R′ is selected from C₁-C₇-alkyl.

As used herein, the term “alkyl” refers to a fully saturated branched or unbranched hydrocarbon moiety having up to 20 carbon atoms. Unless otherwise provided, alkyl refers to hydrocarbon moieties having 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like.

As used herein, the term “acyl chloride” refers to C₁-C₇-alkyl-C(O)—Cl, wherein alkyl is defined as above.

As used herein, the term “acid anhydride” refers to C₁-C₇-alkyl-C(O)—O—C(O)—C₁-C₇-alkyl, wherein alkyl is defined as above.

As used herein, the term “activating group” refers to the respective group resulting from the reaction of a carboxylic acid with an activating agent such as propane phosphonic anhydride; thionyl chloride; oxalyl chloride; 4-(4,6-dimethoxy-1,3,5-triazin-2-yl)-4-methylmorpholinium chloride (DMTMM), or suitable carbodiimides like for example dicyclohexyl-carbodiimide (DCC), N,N′-Diisopropylcarbodiimide (DIC), 1-Ethyl-3-(3-dimethylaminopropyl)carbodiimide (EDC).

The invention relates in a fourth aspect to a method for manufacturing compositions as described herein comprising the step of combining the excipients as described herein to obtain a hydrophilic or hydrophobic matrix, combining the thus obtained matrix with the agent of the invention, and optionally adding an aqueous phase (i.e. a phase containing water and water-soluble excipients).

A composition according to this invention may be prepared by processes that are known per se, but not yet applied for the present compositions where they thus form new processes. In general, the manufacture of a pharmaceutical composition utilizes standard pharmaceutical processes comprising the step of combining the agent of the invention with a matrix, e.g. by mixing, dissolving and/or lyophilizing. Such steps may also comprise heating or cooling the materials used. As outlined above, the agent of the invention is available according to known processes or according to processes as described herein; the individual components of the matrix are either known per se or available according to known processes.

In one embodiment, the invention relates to a method of manufacturing a composition as described in the first aspect of the invention (i.e. a composition of the solution type) comprising the steps of
combining all liquid non-aqueous excipients and the agent of the invention and optionally heating the mixture to 30-95° C. to obtain a solution,
melting the solid excipients at a temperature between 30-95° C. to obtain a melt,
combining the solution and melt, preferably at a temperature between 30-95° C.,
optionally adding water or an aqueous phase to the combined mixture,
optionally cooling down the obtained composition.

In a further embodiment, the invention relates to a method of manufacturing a composition as described in the second aspect of the invention (i.e. a composition of the suspension type) comprising the steps of
combining all excipients at a temperature between 30-95° C. to obtain a melt,
adding the agent of the invention, preferably at a temperature between 30-95° C., to obtain a suspension,
optionally cooling down the obtained composition.

The invention relates in a fifth aspect to the use of 6-(6-hydroxymethyl-pyrimidin-4-yloxy)-naphthalene-1-carboxylic acid (3-trifluoromethyl-phenyl)-amide and compositions thereof in therapeutic applications.

WO 2006/059234 describes certain naphthalene-1-carboxylic acid derivatives, such as 6-(6-hydroxymethyl-pyrimidin-4-yloxy)-naphthalene-1-carboxylic acid (3-trifluoromethyl-phenyl)-amide and various pharmaceutical uses thereof.

Patients suffering from dermatological diseases or conditions, conditions or damages of the retina, or diseases or conditions related to cosmetic dermatology may profit from treatment with a VEGF inhibitor.

Without being bound to theory, it is believed that the agent of the invention is a VEGF inhibitor which is thought to have therapeutic efficacy in the diseases/disorders with a dysregulation/overexpression of VEFG, (neo)-vascularisation, VEGF driven angiogenesis and inflammation.

6-(6-Hydroxymethyl-pyrimidin-4-yloxy)-naphthalene-1-carboxylic acid (3-trifluoromethyl-phenyl)-amide is suitable for the treatment, including prophylaxis and delay of progression, of i) a wide range of dermatological diseases or conditions; ii) cosmetic dermatology.

Compositions comprising 6-(6-hydroxymethyl-pyrimidin-4-yloxy)-naphthalene-1-carboxylic acid (3-trifluoromethyl-phenyl)-amide are suitable for the treatment, including prophylaxis and delay of progression, of i) a wide range of dermatological diseases or conditions; ii) a wide range of diseases, conditions or damages of the retina; iii) cosmetic dermatology.

The term “dermatological diseases” as used herein includes all types of dermatological diseases or conditions in a mammal (preferably a human).

Particularly, 6-(6-hydroxymethyl-pyrimidin-4-yloxy)-naphthalene-1-carboxylic acid (3-trifluoromethyl-phenyl)-amide or a salt, or a polymorph, or a solvate thereof is suitable for the treatment of squamous cell carcinoma, malignant melanoma, Kaposi sarcoma, angiosarcoma, hemangiomas (such as infantile hemangiomas, cutaneous hemangioma, capillary hemangioma, nevus flammeus), lymphangioma, vascular malformations, pyogenic granulomas, angiofibroma, rosacea, dermatitis (such as atopic dermatitis and allergic contact dermatitis), chronic inflammatory skin disorders chronic inflammatory skin disorders (such as bullous diseases) eczema, keloids, diabetic ulcers, lymphedema, actinic keratoses, verrucae vulgares (such as plantar warts), acne and allergic rhinitis/conjunctivitis. More particularly, 6-(6-hydroxymethyl-pyrimidin-4-yloxy)-naphthalene-1-carboxylic acid (3-trifluoromethyl-phenyl)-amide or a salt, or a polymorph, or a solvate thereof is suitable for the treatment of rosacea, dermatitis (such as atopic dermatitis, allergic contact dermatitis), chronic inflammatory skin disorders (such as bullous diseases) eczema, hemangioma (such as cutaneous hemangioma, capillary hemangioma, nevus flammeus) and acne. More particularly, 6-(6-hydroxymethyl-pyrimidin-4-yloxy)-naphthalene-1-carboxylic acid (3-trifluoromethyl-phenyl)-amide or a salt, or a polymorph, or a solvate thereof is suitable for the treatment of rosacea. More particularly, 6-(6-hydroxymethyl-pyrimidin-4-yloxy)-naphthalene-1-carboxylic acid (3-trifluoromethyl-phenyl)-amide or a salt, or a polymorph, or a solvate thereof is suitable for the treatment of erythematotelangiectatic rosecea. More particularly, 6-(6-hydroxymethyl-pyrimidin-4-yloxy)-naphthalene-1-carboxylic acid (3-trifluoromethyl-phenyl)-amide or a salt, or a polymorph, or a solvate thereof is suitable for the treatment of papulopustular rosacea. More particularly, 6-(6-hydroxymethyl-pyrimidin-4-yloxy)-naphthalene-1-carboxylic acid (3-trifluoromethyl-phenyl)-amide or a salt, or a polymorph, or a solvate thereof is suitable for the treatment of phymatous rosacea. More particularly, 6-(6-hydroxymethyl-pyrimidin-4-yloxy)-naphthalene-1-carboxylic acid (3-trifluoromethyl-phenyl)-amide or a salt, or a polymorph, or a solvate thereof is suitable for the treatment of Morbihan disease.

Particularly, the compositions as described herein are suitable for the treatment of squamous cell carcinoma, malignant melanoma, Kaposi sarcoma, angiosarcoma, hemangiomas (such as infantile hemangiomas, cutaneous hemangioma, capillary hemangioma, nevus flammeus), lymphangioma, vascular malformations, pyogenic granulomas, angiofibroma, psoriasis, rosacea, dermatitis (such as atopic dermatitis and allergic contact dermatitis), chronic inflammatory skin disorders chronic inflammatory skin disorders (such as bullous disease) eczema, keloids, diabetic ulcers, lymphedema, actinic keratoses, verrucae vulgares (such as plantar warts) acne and allergic rhinitis/conjunctivitis. More particularly, the compositions as described herein are suitable for the treatment of psoriasis, rosacea, dermatitis (such as atopic dermatitis, allergic contact dermatitis), chronic inflammatory skin disorders (such as bullous diseases) eczema, hemangioma (such as cutaneous hemangioma, capillary hemangioma, nevus flammeus) and acne. More particularly, the compositions are suitable for the treatment of psoriasis, rosacea. More particularly, the compositions are suitable for the treatment of erythematotelangiectatic rosecea. More particularly, the compositions are suitable for the treatment of papulopustular rosacea. More particularly, the compositions are suitable for the treatment of phymatous rosacea. More particularly, the compositions are suitable for the treatment of Morbihan disease.

The term “diseases of the retina” as used herein includes all types of diseases or conditions or damages of the retina of a mammal (preferably a human). Particularly, the compositions as described herein are suitable for the treatment of retinopathy (such as diabetic or hypertensive retinopathy), age related macula degeneration (particularly wet AMD), and macular edema (including diabetic macular edema).

The term “cosmetic dermatology” as used herein includes all types of diseases or conditions or damages of premature skin aging of a mammal (preferably a human), particularly to UV induced premature skin aging of a human and chronically photodamaged skin.

Particularly, 6-(6-hydroxymethyl-pyrimidin-4-yloxy)-naphthalene-1-carboxylic acid (3-trifluoromethyl-phenyl)-amide or a salt, or a polymorph, or a solvate thereof is suitable for the treatment of telangiectasis, wrinkles and/or loss of elastic fibres.

Particularly, the compositions as described herein are suitable for the treatment of telangiectasis, wrinkles and/or loss of elastic fibres.

In one embodiment, the invention relates to the 6-(6-hydroxymethyl-pyrimidin-4-yloxy)-naphthalene-1-carboxylic acid (3-trifluoromethyl-phenyl)-amide or a salt, or a polymorph, or a solvate thereof as pharmaceutical in the treatment of/for use in the treatment of a dermatological disease or condition and/or in cosmetic dermatology selected from squamous cell carcinoma, malignant melanoma, Kaposi sarcoma, angiosarcoma, hemangiomas (such as infantile hemangiomas, cutaneous hemangioma, capillary hemangioma, nevus flammeus), lymphangioma, vascular malformations, pyogenic granulomas, angiofibroma, rosacea, dermatitis (such as atopic dermatitis and allergic contact dermatitis), chronic inflammatory skin disorders chronic inflammatory skin disorders (such as bullous diseases) eczema, keloids, diabetic ulcers, lymphedema, actinic keratoses, verrucae vulgares (such as plantar warts) acne allergic rhinitis/conjunctivitis, telangiectasis, wrinkles and/or loss of elastic fibres.

In a further embodiment, the invention relates to 6-(6-hydroxymethyl-pyrimidin-4-yloxy)-naphthalene-1-carboxylic acid (3-trifluoromethyl-phenyl)-amide or a salt, or a polymorph, or a solvate thereof as pharmaceutical in the treatment of/for use in the treatment of a dermatological disease or condition selected from rosacea, dermatitis (such as atopic dermatitis, allergic contact dermatitis), chronic inflammatory skin disorders (such as bullous diseases) eczema, hemangioma (such as cutaneous hemangioma, capillary hemangioma, nevus flammeus) and acne.

In a further embodiment, the invention relates to 6-(6-hydroxymethyl-pyrimidin-4-yloxy)-naphthalene-1-carboxylic acid (3-trifluoromethyl-phenyl)-amide or a salt, or a polymorph, or a solvate thereof as pharmaceutical in the treatment of/for use in the treatment of rosacea.

In a further embodiment, the invention relates to 6-(6-hydroxymethyl-pyrimidin-4-yloxy)-naphthalene-1-carboxylic acid (3-trifluoromethyl-phenyl)-amide or a salt, or a polymorph, or a solvate thereof as pharmaceutical in the treatment of/for use in the treatment of erythematotelangiectatic rosecea.

In a further embodiment, the invention relates to 6-(6-hydroxymethyl-pyrimidin-4-yloxy)-naphthalene-1-carboxylic acid (3-trifluoromethyl-phenyl)-amide or a salt, or a polymorph, or a solvate thereof as pharmaceutical in the treatment of/for use in the treatment of papulopustular rosacea.

In a further embodiment, the invention relates to 6-(6-hydroxymethyl-pyrimidin-4-yloxy)-naphthalene-1-carboxylic acid (3-trifluoromethyl-phenyl)-amide or a salt, or a polymorph, or a solvate thereof as pharmaceutical in the treatment of/for use in the treatment of phymatous rosacea.

In a further embodiment, the invention relates to 6-(6-hydroxymethyl-pyrimidin-4-yloxy)-naphthalene-1-carboxylic acid (3-trifluoromethyl-phenyl)-amide or a salt, or a polymorph, or a solvate thereof as pharmaceutical in the treatment of/for use in the treatment of Morbihan disease.

In a further embodiment, the invention relates to 6-(6-hydroxymethyl-pyrimidin-4-yloxy)-naphthalene-1-carboxylic acid (3-trifluoromethyl-phenyl)-amide or a salt, or a polymorph, or a solvate thereof for the manufacture of a medicament for the treatment of a dermatological disease or condition and/or in cosmetic dermatology selected from squamous cell carcinoma, malignant melanoma, Kaposi sarcoma, angiosarcoma, hemangiomas (such as infantile hemangiomas, cutaneous hemangioma, capillary hemangioma, nevus flammeus), lymphangioma, vascular malformations, pyogenic granulomas, angiofibroma, rosacea, dermatitis (such as atopic dermatitis and allergic contact dermatitis), chronic inflammatory skin disorders chronic inflammatory skin disorders (such as bullous diseases) eczema, keloids, diabetic ulcers, lymphedema, actinic keratoses, verrucae vulgares (such as plantar warts) acne allergic rhinitis/conjunctivitis, telangiectasis, wrinkles and/or loss of elastic fibres.

In a further embodiment, the invention relates to 6-(6-hydroxymethyl-pyrimidin-4-yloxy)-naphthalene-1-carboxylic acid (3-trifluoromethyl-phenyl)-amide or a salt, or a polymorph, or a solvate thereof for the manufacture of a medicament for the treatment of a dermatological disease or condition selected from rosacea, dermatitis (such as atopic dermatitis, allergic contact dermatitis), chronic inflammatory skin disorders (such as bullous diseases) eczema, hemangioma (such as cutaneous hemangioma, capillary hemangioma, nevus flammeus) and acne.

In a further embodiment, the invention relates to the 6-(6-hydroxymethyl-pyrimidin-4-yloxy)-naphthalene-1-carboxylic acid (3-trifluoromethyl-phenyl)-amide or a salt, or a polymorph, or a solvate thereof for the manufacture of a medicament for the treatment of rosacea.

In a further embodiment, the invention relates to a method of treatment of a dermatological disease or condition; and/or in cosmetic dermatology selected from the group consisting of squamous cell carcinoma, malignant melanoma, Kaposi sarcoma, angiosarcoma, hemangiomas (such as infantile hemangiomas, cutaneous hemangioma, capillary hemangioma, nevus flammeus), lymphangioma, vascular malformations, pyogenic granulomas, angiofibroma, rosacea, dermatitis (such as atopic dermatitis and allergic contact dermatitis), chronic inflammatory skin disorders chronic inflammatory skin disorders (such as bullous diseases) eczema, keloids, diabetic ulcers, lymphedema, actinic keratoses, verrucae vulgares (such as plantar warts) acne allergic rhinitis/conjunctivitis, telangiectasis, wrinkles and/or loss of elastic fibres, which treatment comprises administering to a subject in need of such treatment, particularly a human, an effective amount of 6-(6-hydroxymethyl-pyrimidin-4-yloxy)-naphthalene-1-carboxylic acid (3-trifluoromethyl-phenyl)-amide or a salt, or a polymorph, or a solvate thereof.

In a further embodiment, the invention relates to a method of treatment of a dermatological disease or condition selected from the group consisting of rosacea, dermatitis (such as atopic dermatitis, allergic contact dermatitis), chronic inflammatory skin disorders (such as bullous diseases) eczema, hemangioma (such as cutaneous hemangioma, capillary hemangioma, nevus flammeus) and acne, which treatment comprises administering to a subject in need of such treatment, particularly a human, an effective amount of 6-(6-hydroxymethyl-pyrimidin-4-yloxy)-naphthalene-1-carboxylic acid (3-trifluoromethyl-phenyl)-amide or a salt, or a polymorph, or a solvate thereof.

In a further embodiment, the invention relates to a method of treatment of a dermatological disease or condition selected from rosacea, which treatment comprises administering to a subject in need of such treatment, particularly a human, an effective amount of 6-(6-hydroxymethyl-pyrimidin-4-yloxy)-naphthalene-1-carboxylic acid (3-trifluoromethyl-phenyl)-amide or a salt, or a polymorph, or a solvate thereof.

In a further embodiment, the invention relates to a method as described herein, wherein 6-(6-hydroxymethyl-pyrimidin-4-yloxy)-naphthalene-1-carboxylic acid (3-trifluoromethyl-phenyl)-amide or a salt, or a polymorph, or a solvate thereof for use in the treatment of squamous cell carcinoma, malignant melanoma, Kaposi sarcoma, angiosarcoma, hemangiomas (such as infantile hemangiomas, cutaneous hemangioma, capillary hemangioma, nevus flammeus), lymphangioma, vascular malformations, pyogenic granulomas, angiofibroma, rosacea, dermatitis (such as atopic dermatitis and allergic contact dermatitis), chronic inflammatory skin disorders chronic inflammatory skin disorders (such as bullous diseases) eczema, keloids, diabetic ulcers, lymphedema, actinic keratoses, verrucae vulgares (such as plantar warts) acne and allergic rhinitis/conjunctivitis is administered in combination with another pharmaceutically acceptable composition, either simultaneously or in sequence.

Thus, in another embodiment, the invention relates to a composition as described herein as pharmaceutical/for use as a pharmaceutical. The inventive compositions are particularly suitable and useful in topical, particularly in dermal applications.

In another embodiment, the invention relates to a composition as described herein as pharmaceutical in the treatment of/for use in the treatment of a dermatological disease or condition; a disease, condition or damage of the retina; and/or in cosmetic dermatology.

In a further embodiment, the invention relates to a composition as described herein as pharmaceutical in the treatment of/for use in the treatment of a dermatological disease or condition; a disease, condition or damage of the retina; and/or in cosmetic dermatology, selected from squamous cell carcinoma, malignant melanoma, Kaposi sarcoma, angiosarcoma, hemangiomas (such as infantile hemangiomas, cutaneous hemangioma, capillary hemangioma, nevus flammeus), lymphangioma, vascular malformations, pyogenic granulomas, angiofibroma, psoriasis, rosacea, dermatitis (such as atopic dermatitis and allergic contact dermatitis), chronic inflammatory skin disorders chronic inflammatory skin disorders (such as bullous diseases) eczema, keloids, diabetic ulcers, lymphedema, actinic keratoses, verrucae vulgares (such as plantar warts) acne, allergic rhinitis/conjunctivitis, retinopathy (such as diabetic or hypertensive retinopathy), age related macula degeneration (particularly wet AMD), and macular edema (including diabetic macular edema), telangiectasis, wrinkles and/or loss of elastic fibres.

In a further embodiment, the invention relates to a composition as described herein as pharmaceutical in the treatment of/for use in the treatment of a dermatological disease or condition selected from psoriasis, rosacea, dermatitis (such as atopic dermatitis, allergic contact dermatitis), chronic inflammatory skin disorders (such as bullous diseases) eczema, hemangioma (such as cutaneous hemangioma, capillary hemangioma, nevus flammeus) and acne.

In a further embodiment, the invention relates to a composition as described herein as pharmaceutical in the treatment of/for use in the treatment of a dermatological disease or condition; a disease, condition or damage of the retina; and/or in cosmetic dermatology, particularly in the treatment of/for use in the treatment of psoriasis and/or rosacea.

In a further embodiment, the invention relates to a composition as described herein as pharmaceutical in the treatment of/for use in the treatment of rosacea.

In a further embodiment, the invention relates to a composition as described herein as pharmaceutical in the treatment of/for use in the treatment of erythematotelangiectatic rosacea.

In a further embodiment, the invention relates to a composition as described herein as pharmaceutical in the treatment of/for use in the treatment of papulopustular rosacea.

In a further embodiment, the invention relates to a composition as described herein as pharmaceutical in the treatment of/for use in the treatment of phymatous rosacea.

In a further embodiment, the invention relates to a composition as described herein as pharmaceutical in the treatment of/for use in the treatment of Morbihan disease.

In a further embodiment, the invention relates to a composition as described herein for the manufacture of a medicament for the treatment of a dermatological disease or condition; a disease, condition or damage of the retina; and/or in cosmetic dermatology, particularly in the treatment of/for use in the treatment of psoriasis and/or rosacea.

In a further embodiment, the invention relates to a composition as described herein for the manufacture of a medicament for the treatment of rosacea.

In a further embodiment, the invention relates to a method of treatment of a dermatological disease or condition; a disease, condition or damage of the retina; and/or in cosmetic dermatology (particularly selected from the group consisting of psoriasis and rosacea), which treatment comprises administering to a subject in need of such treatment, particularly a human, an effective amount of a composition as described herein.

In a further embodiment, the invention relates to a method of treatment of a dermatological disease or condition selected from rosacea, which treatment comprises administering to a subject in need of such treatment, particularly a human, an effective amount of a composition as described herein.

In a further embodiment, the invention relates to a composition as described herein as pharmaceutical in the treatment of/for use in the treatment/for the manufacture of a medicament for the treatment of a disease associated with the dysregulation/overexpression of VEGF. The invention also relates to a method of treatment of a disease associated with the dysregulation/overexpression of VEGF, which treatment comprises administering to a subject in need of such treatment, particularly a human, an effective amount of a composition as described herein

In a further embodiment, the invention relates to a method as described herein, wherein a composition as described herein is administered in combination with another pharmaceutically acceptable composition, either simultaneously or in sequence.

For such treatment, the appropriate dosage will, of course, vary depending upon, for example, the chemical nature and the pharmacokinetic data of the agent of the invention employed, the type of composition used, the individual host and the nature and severity of the conditions being treated. However, in general, for satisfactory results in larger mammals, for example humans, an indicated daily dosage is in the range from about 0.01 g to about 1.0 g, of a compound of the present invention; conveniently administered, for example, in divided doses up to four times a day.

The invention relates in a sixth aspect to specific forms of the agent of the invention.

In one embodiment, the invention relates to 6-(6-hydroxymethyl-pyrimidin-4-yloxy)-naphthalene-1-carboxylic acid (3-trifluoromethyl-phenyl)-amide (“agent of the invention”) in crystalline form. Particularly, the invention relates to the crystal forms as defined herein substantially free of other polymorphic forms of the agent of the invention.

In a further embodiment, the invention relates to the agent of the invention in the form of a solvate, particularly a hydrate, such as a hemihydrate. The invention thus relates to a crystal form of the agent of the invention, said crystal additionally contains one or more types of solvent molecules in a stoichiometrically defined amount, preferably one type of solvent molecule, such as water, in the crystal lattice. It was found that hemihydrates have particular beneficial properties: they are stable modifications under ambient conditions and in solutions containing water. Hemihydrates are considered particularly suitable for the manufacturing of the compositions as described herein.

In a further embodiment, the invention relates to the agent of the invention in form of a hemihydrate (Crystal form A), comprising the following X-ray powder diffraction peaks at 7.4, 9.9 and 11.1° 2 Theta. A characteristic line in the X-ray diffraction diagram can be observed at an angle of diffraction 2theta of 24.8° having a strong intensity. Further characteristic lines can be observed e.g. at 7.4, 9.9, 11.1, 14.9 and 15.8° 2 Theta by reflection technique.

The characteristic line at 15.8 is found to be crystal form specific by transmission technique More broadly by transmission technique, the form A can be characterized by one or several of diffractions peaks at angles of diffraction 2theta of 2.2, 6.6, 15.8, 19.4° 2 Theta.

In a further embodiment, the invention relates to the agent of the invention in form of a hemihydrate (Crystal form B), comprising the following X-ray powder diffraction peaks (transmission technique):

In a further embodiment, the invention relates to the agent of the invention in form of a hemihydrate (Crystal form B), comprising the following X-ray powder diffraction peaks at 4.4, 6.6 and 11.1° 2 Theta. A characteristic line in the X-ray diffraction diagram can be observed at an angle of diffraction 2theta of 18.1° having a strong intensity. Further characteristic lines can be observed e.g. at 2.2, 4.4, 6.6, 11.1, 13.3 and 18.1° 2 Theta by reflection technique. The characteristic line at 12.3 is found to be crystal form specific by transmission technique despite its weak intensity compared to the other lines.

More broadly by transmission technique, the form B can be characterized by one or several of diffractions peaks at angles of diffraction 2theta of 2.2, 11.1, 12.3, 16.6 and 20.4° 2 Theta.

Relative intensities are dependent on several factors including particle size, shape and method of sample preparation, thus are subject to variation. They have been included for information only and are in no way intended to limit the invention. 2-theta values herein have an error range +/−0.2.

It was found that Crystal form B is a particularly stable modification under ambient conditions and therefore preferred for the manufacturing of the compositions as described herein.

### Form B in Transmission

| Angle 2- | |
|---|---|
| Theta | Intensity |
| 2.2 | high |
| 4.4 | Low |
| 6.6 | Low |
| 11.1 | Medium |
| 12.3 | Low |
| 13.3 | Low |
| 16.6 | Medium |
| 18.2 | Medium |
| 18.7 | Medium |
| 19.0 | Medium |
| 19.2 | Medium |
| 19.7 | Medium |
| 20.1 | Medium |
| 20.4 | Medium |
| 21.4 | Medium |
| 22.0 | Medium |
| 23.2 | Medium |
| 23.6 | Medium |
| 24.8 | Medium |
| 25.1 | High |
| 25.3 | Medium |

### Form B in Reflexion:

| Angle 2- | |
|---|---|
| Theta, | Intensity |
| 2.2 | Medium |
| 4.4 | Low |
| 6.6 | Medium |
| 11.1 | Medium |
| 13.3 | Medium |
| 16.5 | Medium |
| 16.8 | Medium |
| 17.5 | Medium |
| 18.6 | Medium |
| 19.1 | High |
| 19.7 | High |
| 20.0 | High |
| 20.4 | High |

### Form A in Transmission

| Angle 2- | |
|---|---|
| Theta, | Intensity |
| 2.2 | High |
| 6.6 | Medium |
| 15.8 | Medium |
| 16.7 | High |
| 16.9 | Medium |
| 18.2 | High |
| 18.4 | Medium |
| 18.9 | High |
| 19.4 | High |
| 19.6 | High |
| 20.0 | Medium |
| 20.2 | High |
| 20.7 | Medium |
| 21.1 | Medium |
| 21.8 | Medium |
| 22.1 | Medium |
| 24.8 | High |
| 25.1 | High |
| 25.6 | Medium |
| 25.9 | Medium |
| 26.2 | Medium |
| 27.1 | Medium |

### Form A in Reflexion:

| Angle 2- | |
|---|---|
| Theta, | Intensity % |
| 2.3 | High |
| 6.7 | Medium |
| 7.4 | Low |
| 9.9 | Low |
| 11.1 | Medium |
| 13.4 | Medium |
| 14.9 | Medium |
| 15.8 | Medium |
| 16.6 | Medium |
| 16.9 | Medium |
| 17.3 | Medium |
| 18.2 | High |
| 19.0 | High |
| 19.4 | High |
| 20.1 | High |
| 20.7 | Medium |
| 21.1 | High |
| 21.8 | High |
| 22.1 | High |
| 22.4 | Medium |
| 23.0 | Medium |
| 23.5 | High |
| 24.0 | Medium |
| 24.8 | High |
| 25.1 | High |
| 25.6 | High |

### Form B in Reflexion (Highly Crystalline Material)

| Angle 2- | |
|---|---|
| Theta, | Intensity % |
| 2.3 | High |
| 4.5 | Medium |
| 6.7 | High |
| 8.9 | Medium |
| 11.1 | High |
| 13.4 | High |
| 17.9 | Medium |
| 20.1 | High |
| 22.4 | Medium |
| 24.7 | Medium |
| 26.9 | High |
| 29.2 | Medium |
| 31.5 | High |

In a further embodiment, the invention relates to a method of manufacturing crystalline forms of the agent of the invention and/or a method of purifying the agent of the invention, comprising the step of crystallizing the agent of the invention from a solution containing or consisting C1-C4 alcohol. Suitable starting materials for such method include the agent of the invention a) in crude form (i.e. containing impurities) or b) amorphous form or c) in an undesired crystalline form.

Advantageously, such method comprises the steps of
dissolving the agent of the invention in an C1-C4 alcohol which may contain up to 30 wt. % of additional solvents, at elevated temperatures, such as reflux temperature,
crystallizing the solution at reduced temperatures, such as −5° C.-+35° C., optionally by adding seed crystals,
separating the obtained crystals of the agent of the invention,
removing solvent under reduced pressure to obtain pure crystalline agent of the invention or a solvate thereof.

In a further embodiment, the invention relates to a method of manufacturing hemihydrates of the agent of the invention, comprising the steps of
dissolving the agent of the invention in an C1-C4 alcohol which may contain up to 30 wt. % water at elevated temperatures, such as reflux temperature,
effecting crystallizing at reduced temperatures, such as −5° C.-+35° C., optionally by adding seed crystals,
separating the obtained crystals of the agent of the invention
removing solvent at low temperatures and under mild vacuum, e.g. below 50° C., 30 mbar, until the water content is in the range between of 2.2% and 3.0%, to obtain the agent of the invention as hemihydrate;
Alternatively, the last step can be replaced by removing solvent under reduced pressure followed by rehydration, to obtain the agent of the invention as hemihydrate.

The purification/manufacturing process of the agent of the invention may be described as follows: Step 1: The crude agent of the invention is mixed with a C1-C4 alcohol which optionally contains up to 30% wt water. Preferred alcohols are methanol, ethanol, n-propanol and iso-propanol, particularly preferably ethanol. (Presence of a certain amount of water, which is a practically anti-solvent of the drug substance, in the mentioned solvent can decrease the solubility of the drug substance to a proper value which enables commercializing the process. On the other hand, water is necessary for the formation of the desired hydrate.)

Step 2: The obtained mixture is refluxed to obtain a clear solution. Optionally, a clear filtration is conducted. If at the beginning the drug substance is dissolved in pure solvent, or in the solvent containing less than the desired amount of water, additional water may be charged into the clear solution to reach the desired water content, as long as the solution remains clear without any precipitate.

Step 3: The obtained solution is then slowly cooled down to obtain a meta-stable solution; e.g. to 50±5° C. with a cooling rate of approximately 0.5° C./min.

Step 4: Crystallization is initiated, e.g. by addition of seed crystals. This induces a controlled crystallization process in order to have desired form, crystal structure and morphology. The seeded-crystallization can also minimize the occurrence of sudden precipitation which to a large extent accounts for the formation of fine particles and for bad purification effect due to inclusion of impurity species in the crystals. The seed crystals prepared, for instance, by milling the coarse material, should be fine particles with narrow particle size distribution. The quantity of seed material can be 0.01%-1% wt of the crude agent of the invention. After seeding the solution turns to turbid suspension and after holding for a certain time at constant temperature it remains turbid.

Step 5: The system is further cooled down, e.g. to 0-5° C. with a cooling rate of approx. 0.1° C./min or less. Slowly cooling assures a slow to moderate crystal growth rate which is crucial to obtain crystals with desired structure and purity.

Step 6: The thus obtained suspension is filtrated and the wet material on the filter is washed with alcohol/H₂O mixture (ratio 1:1) for 2-3 times. Optionally the filter cake is further washed 1-2 times with pure H₂O.

Step 7: The isolated wet material is dried at low temperatures and under mild vacuum, e.g. below 50° C., ≧30 mbar, until the water content is in the range of 2.2% and 3.0%. In case of overdrying, rehydration step is carried out in Rh range of 20 to 90% for fixed time to regain hemihydrate crystalline form B. Crystalline hemihydrate, polymorph B of the compound of the invention is thus obtained and confirmed by XRPD, TGA and Karl-Fischer titration.

In a further embodiment, the invention relates to the agent of the invention obtainable by or obtained by a process as described herein.

### MODES FOR CARRYING OUT THE INVENTION

The following Examples serve to illustrate the invention without limiting the scope thereof. It is understood that the invention is not limited to the embodiments set forth herein, but embraces all such forms thereof as come within the scope of the disclosure.

Temperatures are given in degrees Celsius (°). Unless otherwise indicated, the reactions take place at room temperature under N₂-atmosphere. The R_{f} values which indicate the ratio of the distance moved by each substance to the distance moved by the eluent front are determined on silica gel thin-layer plates (Merck, Darmstadt, Germany) by thin-layer chromatography using the respective named solvent systems.

### Abbreviations:

Anal. elemental analysis (for indicated atoms, difference between calculated and measured values ≦0.4%)
brine saturated solution of NaCl in H₂O
conc. concentrated
DEPC diethyl-cyanophosphonate
DIPE diisopropyl-ether
DMAP dimethylaminopyridine
DMTMM 4-(4,6-dimethoxy-1,3,5-triazin-2-yl)-4-methylmorpholinium chloride
eq. equivalent
HATU O-(7-azabenzotriazol-1-yl)-N,N,N′,N′-tetramethyluronium-hexafluorophosphate
HPC hydroxypropyl cellulose
HPLC high pressure liquid chromatography
ICH International Conference on Harmonization
m.p. melting point
MPLC medium pressure liquid chromatography (Combi Flash system: normal phase SiO₂; Gilson system: reversed phase Nucleosil C18 (H₂O/CH₃CN+TFA), product obtained as free base after neutralization with NaHCO₃)
MS mass spectrum
NMM N-methyl-morpholine
NMP N-methyl-pyrrolidone
prep-HPLC preparative high pressure liquid chromatography; Waters system; column: reversed phase Atlantis™ (100×19 mm), dC18 OBD (H₂O/CH₃CN+0.1% TFA), 5 μM, generally product obtained as a TFA salt after lyophilization.
propylphosphonic anhydride N-propylphosphonic acid anhydride, cyclic trimer[68957-94-8]; 50% in DMF
R_{f} ratio of fronts (TLC)
rt room temperature
sat. saturated
THF tetrahydrofuran (distilled from Na/benzophenone)
TFA trifluoroacetic acid
TLC thin layer chromatography
t_{Ref} retention time (HPLC)
triphosgene bis(trichloromethyl) carbonate

“Mod”, or “modification” herein is also referred to herein as “crystal form”.

### A Agent of the Invention

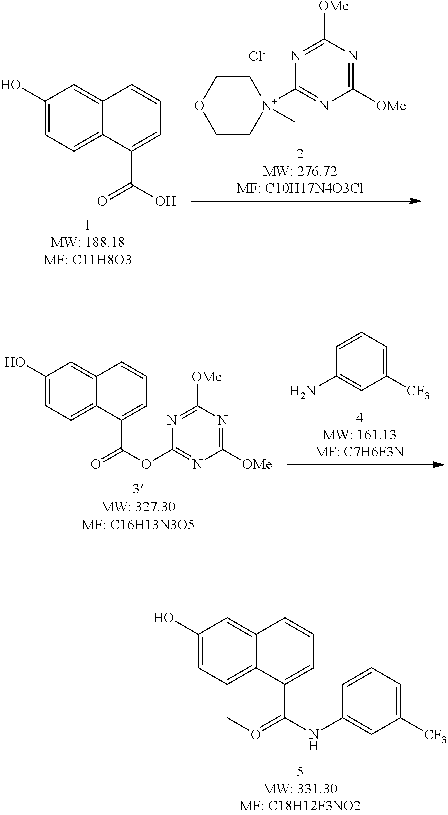

### Example A

### 6-Hydroxy-naphthalene-1-carboxylic acid 4,6-dimethoxy-[1,3,5]triazin-2-yl ester

6-Hydroxy-naphthalene-1-carboxylic acid (65.0 g, 1.0 eq) was suspended at 20° C. in acetonitrile (975 ml). The suspension was cooled down to 10° C. and DMTMM (105 g, 1.1 eq) was added over a period of 30-60 min, maintaining the temperature at 10-15° C. After stirring the mixture at 20° C. for 15 h, water (975 ml) was added over a period of 30-60 min. The resulting suspension was stirred at 20° C. for 3 h and the solids were collected by filtration. The filter cake was washed with water and was dried at 50° C. under full vacuum to give 6-hydroxy-naphthalene-1-carboxylic acid 4,6-dimethoxy-[1,3,5]triazin-2-yl ester (96.1 g, 85% of theory).

1H-NMR (DMSO-d6): 10.16 (1H); 8.73 (1H); 8.18 (2H); 7.59 (1H); 7.33 (2H); 4.01 (6H). MS (ESI, m/e) 326 [M−H]−. mp.: 166-168° C.

### Example B

### 6-Hydroxy-naphthalene-1-carboxylic acid (3-trifluoromethyl-phenyl)-amide

6-Hydroxy-naphthalene-1-carboxylic acid 4,6-dimethoxy-[1,3,5]triazin-2-yl ester (60.0 g, 1.0 eq) was then dissolved in N-methyl-2-pyrrolidinone (185 ml) at 20° C. To the resulting solution, 3-trifluoromethyl-phenylamine [CAS 98-16-8] (44.3 g, 1.5 eq) was added over a period of 30 min. The mixture was then heated to 55° C. for 16 h and was then cooled down to 22° C. After addition of ethyl acetate (600 ml), the mixture was stirred at 22° C. for another 60 min. The mixture was then filtered and the filter cake was washed with ethyl acetate (60 ml). The layers of the combined filtrates were separated and the organic layer was washed with 2 N HCl solution, water, aqueous sodium hydrogen carbonate solution and aqueous sodium chloride solution. The organic layer was partially concentrated at 40° C. under reduced pressure and toluene (600 ml) was added at 60° C. over 1-2 h. The suspension was partially concentrated under reduced pressure at 60° C. and toluene (300 ml) was added at 40° C. After heating the suspension to 80° C. for 30 min, the mixture was cooled to 20° C. within 6 h and the precipitating solids were isolated by filtration. The filter cake was washed with toluene and was dried at 50° C. to give 6-hydroxy-naphthalene-1-carboxylic acid (3-trifluoromethyl-phenyl)-amide (46.2 g, 76% of theory) as off-white powder.

1H-NMR (DMSO-d6): 10.82 (1H); 9.90 (1H); 8.32 (1H); 8.05 (1H); 7.98 (1H); 7.85 (1H); 7.60 (1H); 7.53 (1H); 7.50 (1H); 7.46 (1H); 7.21 (1H); 7.15 (1H). MS (ESI, m/e) 332 [M+H]+. mp.: 201-202° C. IR (v/cm-1): 3267, 3094, 2707, 1639, 1557, 1439, 1332, 1166, 1122, 793.

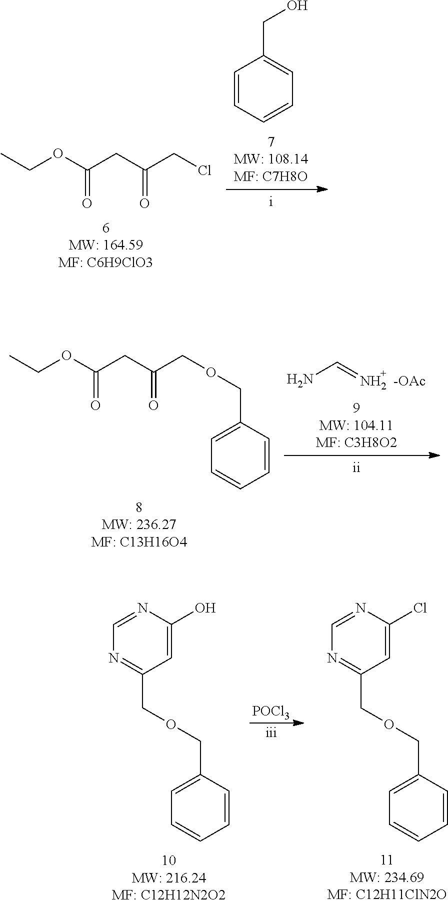

### Example C

### 4-Benzyloxy-3-oxo-butyric acid ethyl ester [CAS 67354-34-1]

Sodium hydride (23.9 g, 2.0 eq) was suspended in tetrahydrofuran (280 g) at ambient temperature. The mixture was then cooled to 15° C. and phenyl-methanol [CAS 185532-71-2] (32.4 g, 1.0 eq) was added within 30 min while maintaining the temperature below 15° C. To the resulting solution, 4-chloro-3-oxo-butyric acid ethyl ester [CAS 638-07-3] (49.4 g, 1.0 eq) was added within 30 min while maintaining the temperature below 15° C. The solution was then stirred at 80° C. for 18 h and was subsequently cooled down to 15° C. Water (100 ml) was added and the mixture was partially concentrated under vacuum at 30° C. to remove tetrahydrofuran. Aqueous citric acid solution was added and the reaction mixture was extracted with isopropyl acetate. The combined organic layers were washed with water and were concentrated at 30° C. under reduced pressure to give 4-benzyloxy-3-oxo-butyric acid ethyl ester (70.0 g, 99% of theory) as yellow oil.

1H-NMR (CDCl3): 7.37 (?H); 4.60 (?H); 4.13 (?H); 3.51 (?H); 1.26 (?H).

### Example D

### 6-Benzyloxymethyl-pyrimidin-4-ol [CAS 188177-37-9]

Sodium methoxide (67.8 g of a 30% solution in methanol, 2.5 eq) was added to methanol (320 g) at ambient temperature. The mixture was cooled down to 5° C., formamidine acetate, [CAS 3473-63-0] 23.4 g, 1.5 eq) was added followed by the addition of compound 8 (35.4 g, 1.0 eq in 50 ml methanol) within 30 min while maintaining the temperature below 5° C. The mixture was stirred at 22° C. for 3 d and was then partially concentrated at 30° C. under reduced pressure to remove methanol. Water and aqueous citric acid solution was added and the reaction mixture was extracted with ethyl acetate. The combined organic layers were washed with water and were concentrated at 30° C. under reduced pressure. Isopropyl acetate was added at and the precipitating solids were collected by filtration. The filter cake was washed with isopropyl acetate and was dried at 22° C. under vacuum to give 6-benzyloxymethyl-pyrimidin-4-ol (25.9 g, 80% of theory) as white solid.

1H-NMR (DMSO-d6): 13.39 (1H); 8.12 (1H); 7.38-7.26 (5H); 6.70 (1H); 4.67 (2H); 4.45 (2H). MS (ESI, m/e) 217 [M+H]+. mp.: 102-103° C.

### Example E

### 4-Benzyloxymethyl-6-chloro-pyrimidine [CAS 914802-11-2]

6-Benzyloxymethyl-pyrimidin-4-ol (10.8 g, 1.0 eq) was suspended in toluene (150 ml) at ambient temperature. To the resulting suspension, phosphorous oxychloride (30.6 g, 4.0 eq) and tripropylamine (21.3 g, 3.0 eq) were added. The reaction mixture was then stirred at 40° C. for 1 h. At 5° C., an aqueous solution of ammonium hydroxide was added and the phases of the resulting emulsion were separated. The organic layer was washed with water and was then passed through a pad of silica gel. The filtrate was concentrated at 30° C. under reduced vacuum to obtain 4-benzyloxymethyl-6-chloro-pyrimidine (8.19 g, 70% of theory) as pale yellow oil.

1H-NMR (DMSO-d6): 8.98 (1H); 7.64 (1H); 7.40-7.29 (5H); 4.65 (4H). MS (ESI, m/e) 235 [M+H]+. IR (v/cm-1): 3032; 2863; 1569; 1536; 1454; 1318; 1111; 1091; 904; 744.

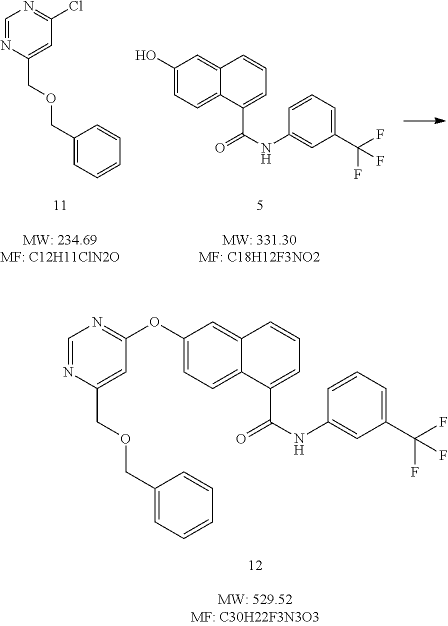

### Example F

### 6-(6-Benzyloxymethyl-pyrimidin-4-yloxy)-naphthalene-1-carboxylic acid (3-trifluoromethyl-phenyl)-amide

To a suspension of 6-hydroxy-naphthalene-1-carboxylic acid (3-trifluoromethyl-phenyl)-amide (16.6 g, 1.0 eq) and potassium carbonate (20.7 g, 3.0 eq) in N-methyl-2-pyrrolidone (40 ml) at 100° C., a solution of compound 11 (12.3 g, 1.05 eq) in N-methyl-2-pyrrolidone (15.0 ml) was added within approx. 60 min. The reaction mixture was then cooled down to 22° C., isopropyl acetate (220 ml) and aqueous sodium chloride solution (220 ml, 10% m/m solution) were added. The organic layer was then washed using aqueous citric acid solution (216 ml, 5% m/m solution) and water (110 ml). Subsequently, the solvent of the organic layer was separated, was concentrated to approx. half of its initial volume at 40° C. under reduced pressure and toluene (150 ml) was added. The resulting mixture was again concentrated at 40° C. under reduced pressure and toluene (150 ml) was added. The resulting suspension was cooled down to 0-5° C. and the solids were isolated by filtration. The filter cake was washed with toluene and was dried at 55° C. under full vacuum to give 6-(6-benzyloxymethyl-pyrimidin-4-yloxy)-naphthalene-1-carboxylic acid (3-trifluoromethyl-phenyl)-amide (23.1 g, 87% of theory) as fine, off-white solid.

1H-NMR (DMSO-d6): 10.96 (1H); 8.72 (1H); 8.33 (2H); 8.11 (1H); 8.03 (1H); 7.92 (1H); 7.84 (1H); 7.70-7.62 (2H); 7.50 (2H); 7.36-7.27 (5H); 7.16 (1H); 4.65 (2H); 4.63 (2H). MS (ESI, m/e) 530 [M+H]+. mp.: 123-124° C. IR (v/cm-1): 3269; 3026; 2864; 1650; 1553; 1370; 1337; 1169; 1129; 700.

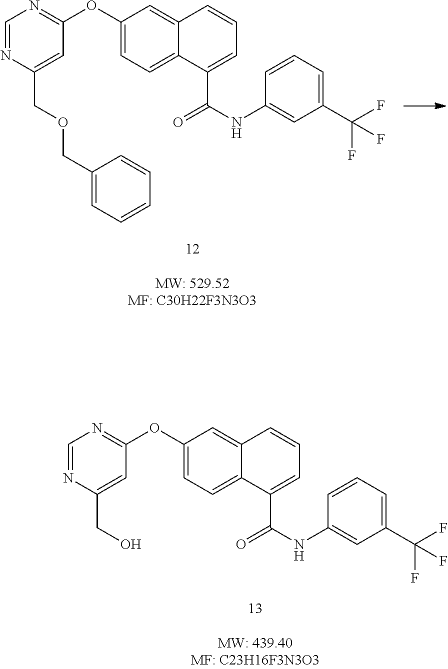

### Example G (Conditions a)

### 6-(6-Hydroxymethyl-pyrimidin-4-yloxy)-naphthalene-1-carboxylic acid (3-trifluoromethyl-phenyl)-amide

6-(6-Benzyloxymethyl-pyrimidin-4-yloxy)-naphthalene-1-carboxylic acid (3-trifluoromethyl-phenyl)-amide (20.0 g, 1.0 eq) was heated in a mixture of trifluoroacetic acid (108 g, 25 eq) and toluene (10 ml) at 70° C. for 17 h. The reaction mixture was then cooled down to 22° C. and was subsequently quenched through addition to a mixture of aqueous 3 M sodium hydroxide solution (300 g) and sodium chloride (55.0 g) at 5-10° C. The pH of the resulting solution was then adjusted to 6-9 by addition of aqueous 3 N sodium hydroxide solution (22 ml). To the resulting suspension, 2-methyltetrahydrofurane (240 ml) was added and the mixture was stirred at 30° C. until all solids dissolved. The phases were separated and the organic layer was treated optionally with activated charcoal, was optionally filtered over aluminum oxide and was washed with aqueous sodium hydrogen carbonate solution and water. Finally, the organic layer was partially concentrated at 40° C. under reduced pressure and toluene (150 ml) was added. The resulting suspension was cooled down to 22° C. and the solids were isolated by filtration. The filter cake was washed with toluene and was dried at 50° C. under full vacuum to give 6-(6-hydroxymethyl-pyrimidin-4-yloxy)-naphthalene-1-carboxylic acid (3-trifluoromethyl-phenyl)-amide (14.4 g, 87% of theory) as fine, off-white solid.

### Example G (Conditions b)

### 6-(6-hydroxymethyl-pyrimidin-4-yloxy)-naphthalene-1-carboxylic acid (3-trifluoromethyl-phenyl)-amide

6-(6-Benzyloxymethyl-pyrimidin-4-yloxy)-naphthalene-1-carboxylic acid (3-trifluoromethyl-phenyl)-amide (10.0 g, 1.0 eq) was suspended in dichloromethane (50.0 ml) at ambient temperature. The suspension was cooled to −5-0° C. and methanesulfonic acid (36.3 g, 20.0 eq) were added within 90 min while maintaining the temperature between −5-5° C. The solution was then heated to 20° C. and the solution was agitated at 20° C. for 8 h. The reaction mixture was then cooled to −5-0° C. and 2 M aqueous sodium hydroxide solution was added (133 ml). After agitation at 20° C. for 2 h, the suspension was filtered and the filter cake was washed with water and ethanol. The isolated material was dried at 50° C. in vacuo to give 6-(6-hydroxymethyl-pyrimidin-4-yloxy)-naphthalene-1-carboxylic acid (3-trifluoromethyl-phenyl)-amide (7.5 g, 90% of theory) as off-white solid.

1H-NMR (DMSO-d6): 10.96 (1H); 8.67 (1H); 8.34 (2H); 8.11 (1H); 8.02 (1H); 7.91 (1H); 7.83 (1H); 7.70-7.61 (2H); 7.50 (2H); 7.12 (1H); 5.68 (1H); 4.56 (2H). MS (ESI, m/e) 440 [M+H]+. IR (v/cm-1): 3281; 3065; 2852; 1650; 1553; 1372; 1337; 1166; 1132; 700.

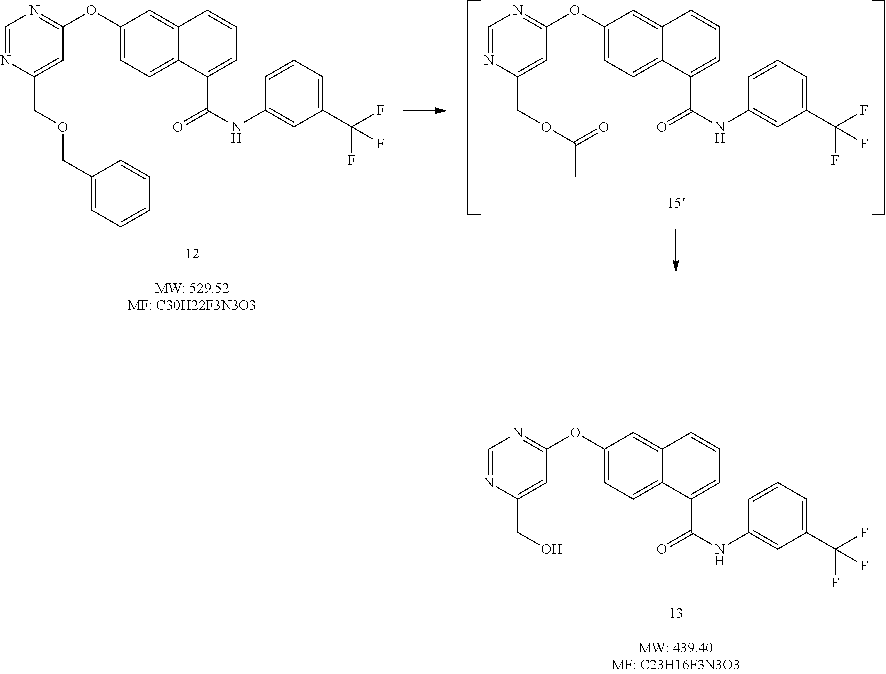

### Example H

### 6-(6-Hydroxymethyl-pyrimidin-4-yloxy)-naphthalene-1-carboxylic acid (3-trifluoromethyl-phenyl)-amide

6-(6-Benzyloxymethyl-pyrimidin-4-yloxy)-naphthalene-1-carboxylic acid (3-trifluoromethyl-phenyl)-amide (10.0 g, 1.0 eq) was suspended in acetic anhydride (16.2 g, 8.4 eq) at ambient temperature. The suspension was then heated to 70° C., sulfuric acid 97% (5.52 g=2.9 eq) was added and the mixture was agitated at 70° C. for 1 h. The reaction mixture was then cooled down to 40° C. and was subsequently quenched through addition to aqueous 3 M sodium hydroxide solution (124 ml) while maintaining the temperature below 20° C. To the resulting mixture, 2-methyltetrahydrofurane (75 ml) was added at 30° C., the organic layer was separated and was washed with aqueous sodium hydrogen carbonate solution (25 ml). The organic layer was then treated optionally with activated charcoal and was optionally filtered over aluminum oxide. The solution containing acetic acid 6-[5-(3-trifluoromethyl-phenylcarbamoyl)-naphthalen-2-yloxy]pyrimidin-4-ylmethyl ester was subsequently heated to 50° C. and methanol (20 ml) and sodium methoxide (0.150 ml of a 30% solution in methanol, 0.04 eq) were added. The mixture was stirred at 50° C. for 5 h and 2-methyltetrahydrofurane and water were added. After phase separation at 20° C., the organic layer was washed with water and was partially concentrated at 40° C. under reduced pressure. Toluene was added and the resulting suspension was cooled down to 22° C. and the solids were isolated by filtration. The filter cake was washed with toluene and was dried at 50° C. under full vacuum to give 6-(6-hydroxymethyl-pyrimidin-4-yloxy)-naphthalene-1-carboxylic acid (3-trifluoromethyl-phenyl)-amide (7.08 g, 85% of theory) as fine, off-white solid.

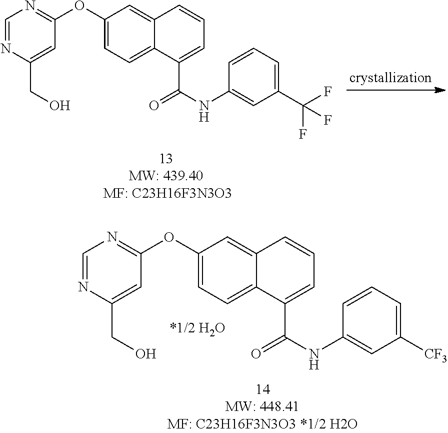

### Example I

### 6-(6-Hydroxymethyl-pyrimidin-4-yloxy)-naphthalene-1-carboxylic acid (3-trifluoromethyl-phenyl)-amide hemihydrate

6-(6-Hydroxymethyl-pyrimidin-4-yloxy)-naphthalene-1-carboxylic acid (3-trifluoromethyl-phenyl)-amide (9.0 g) was dissolved in a mixture of ethanol (87.3 ml) and water (7.6 ml) at 65° C. The solution was filtered hot and was then cooled down to 55° C. At 55° C., seed suspension was added to the solution to induce crystallization. The suspension was linearly cooled down to 0° C. within 8 h, and the precipitating solids were collected by filtration. The filter cake was washed with a mixture of ethanol and water and was dried at 40° v under reduced pressure to give 6-(6-hydroxymethyl-pyrimidin-4-yloxy)-naphthalene-1-carboxylic acid (3-trifluoromethyl-phenyl)-amide hemihydrate (7.5 g, 83% of theory) as hemihydrate in form of white crystals.

1H-NMR (DMSO-d6): 10.96 (1H); 8.67 (1H); 8.34 (2H); 8.11 (1H); 8.02 (1H); 7.91 (1H); 7.83 (1H); 7.70-7.62 (2H); 7.49 (2H); 7.12 (1H); 5.69 (111; 4.56 (2H). MS (ESI, m/e) 440 [M+H]+. mp.: 182° C. IR (v/cm-1): 3281; 3065; 2851; 1650; 1553; 1372; 1337; 1165; 1131; 700.

### Process Described in WO 2006/059234

### Example 1

### 6-(6-Hydroxymethyl-pyrimidin-4-yloxy)-naphthalene-1-carboxylic acid (3-trifluoromethyl-phenyl)-amide

To 1.16 g (2.41 mMol) 6-[5-(3-trifluoromethyl-phenylcarbamoyl)-naphthalen-2-yloxy]-pyrimidine-4-carboxylic acid ethyl ester in 40 ml tert-butanol, 218 mg (5.76 mMol) NaBH₄ are added and the mixture is stirred for 1 h at 70° C. Then additional 109 mg NaBH₄ are added and stirring is continued for another 1 h at 80° C. The reaction mixture is concentrated in vacuo and the residue re-dissolved in EtOAc and sat. NaHCO₃. The separated aqueous phase is extracted twice with EtOAc. The organic layers are washed with sat. NaHCO₃ and brine, dried (Na₂SO₄) and concentrated after addition of SiO₂. This powder is put on top of a SiO₂-column (CH₂Cl₂/EtOAc 2:1→1:1→EtOAc): At first the side product 6-(6-methyl-pyrimidin-4-yloxy)-naphthalene-1-carboxylic acid (3-trifluoromethyl-phenyl)-amide is eluated {MS: [M+1]⁺=424; TLC(CH₂Cl₂/EtOAc 1:1) R_{f}=0.33; HPLC: ^{A}t_{Ref}=15.1}, followed by the title compound: m.p.: 183-184° C.; MS: [M+1]⁺=440; TLC(CH₂Cl₂/EtOAc 1:1): R_{f}=0.13; HPLC: ^{A}t_{Ref}=14.3; Anal.: C, H, N, F.

HPLC Conditions: ^{A}t_{Ref}: retention time [min] for System A: Linear gradient 20-100% CH₃CN (0.1% TFA) and H₂O (0.1% TFA) in 13 min+5 min 100% CH₃CN (0.1% TFA); detection at 215 nm, flow rate 1 ml/min at 25 or 30° C. Column: Nucleosil 120-3 C18 (125×3.0 mm).

### Example 2

### 6-(6-Hydroxymethyl-pyrimidin-4-yloxy)-naphthalene-1-carboxylic acid (3-trifluoromethyl-phenyl)-amide hemihydrate, modification B

### Synthesis:

In a 250 ml glass reactor 9 g crude 6-(6-hydroxymethyl-pyrimidin-4-yloxy)-naphthalene-1-carboxylic acid (3-trifluoromethyl-phenyl)-amide was dissolved in 81 g mixture of ethanol/water (ratio=9:1) at 65° C. After cooling to 55° C. 9 mg seed crystals (6-(6-Hydroxymethyl-pyrimidin-4-yloxy)-naphthalene-1-carboxylic acid (3-trifluoromethyl-phenyl)-amide Mod. B, micronized) were added to induce crystallization. The turbid solution was cooled to 0° C. within 8 hours. The suspension was isolated on a filter frit and the wet product was washed 3 times with 20 g mixture of ethanol/water (1:1) and then further washed twice with 20 g pure water. The wet product was dried in an oven at 40° C. and 30 mbar for 17 hours; 7.50 g white product was obtained.

### Analysis:

Karl-Fischer titration of the resultant product showed a water content of 2.80%.

TGA analysis confirmed the product was 6-(6-hydroxymethyl-pyrimidin-4-yloxy)-naphthalene-1-carboxylic acid (3-trifluoromethyl-phenyl)-amide hemihydrate, Mod. B.

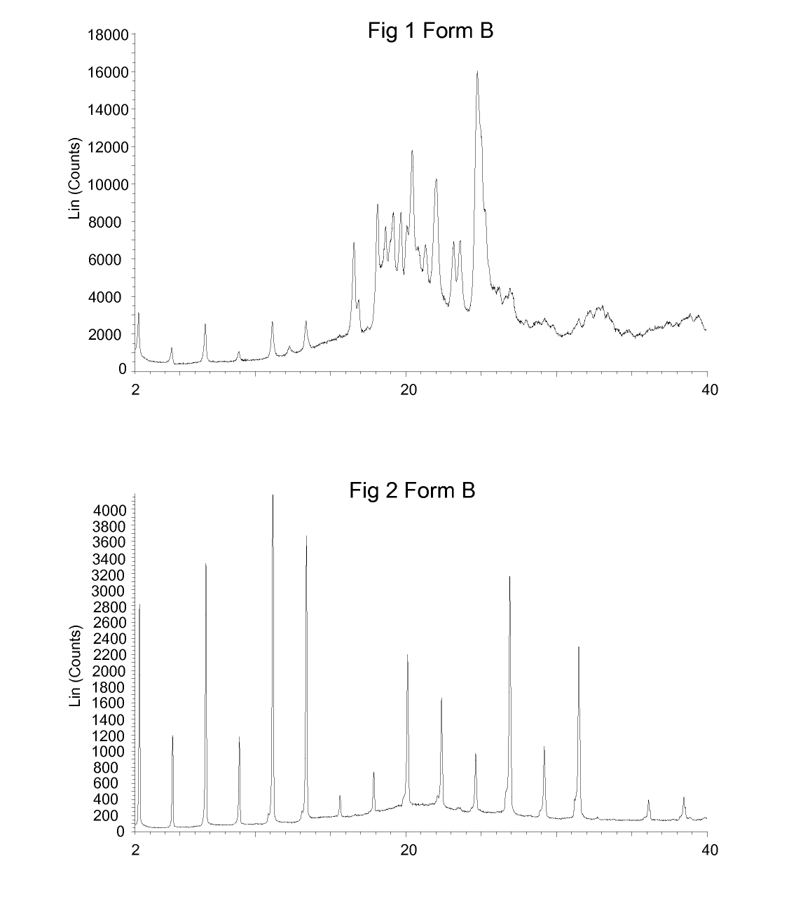

XRPD analysis was performed as described below; this also confirmed the product was 6-(6-hydroxymethyl-pyrimidin-4-yloxy)-naphthalene-1-carboxylic acid (3-trifluoromethyl-phenyl)-amide hemihydrate, Mod. B. FIG. 1 shows the obtained XRPD pattern in reflexion geometry; the background contribution is due to a kapton foil which is used to protect the sample. The instrument parameters were as follows: Bruker D8 Advance X-Ray diffractometer, Mode reflexion, Scan range 2°-40° (2 theta value), CuKα (45 kV, 40 mA). It was further observed that if the drug substance is not milled, some strong preferential orientation phenomena are observed, when using the same instrument parameters. It is believed that the pattern might be evaluated differently, but if grinded, it corresponds to modification B. FIG. 4 shows the obtained XRPd pattern in transmission geometry. The instrument parameters were as follows: Bruker D8 Vario X-Ray diffractometer, Mode Transmission, Scan range 2°-40° (2 theta value), CuKα (45 kV, 40 mA). Temperature: 20 Degrees C.

### Example 3

### 6-(6-Hydroxymethyl-pyrimidin-4-yloxy)-naphthalene-1-carboxylic acid (3-trifluoromethyl-phenyl)-amide hemihydrate, modification B

Similar as Example 2 but starting with 12 g crude 6-(6-hydroxymethyl-pyrimidin-4-yloxy)-naphthalene-1-carboxylic acid (3-trifluoromethyl-phenyl)-amide were dissolved in 88 g of a mixture of n-propanol/water (ratio=9.8:0.2) and followed the same crystallization procedure. After filtration of the crystal suspension, the wet product was washed 3 times with mixture of n-propanol/water (ratio=1:1). The material was then dried at 40° C. and 30 mbar for 24 hours. XRPD and TGA analysis showed the product was 6-(6-hydroxymethyl-pyrimidin-4-yloxy)-naphthalene-1-carboxylic acid (3-trifluoromethyl-phenyl)-amide hemihydrate Mod. B.

### Example 4

### 6-(6-Hydroxymethyl-pyrimidin-4-yloxy)-naphthalene-1-carboxylic acid (3-trifluoromethyl-phenyl)-amide hemihydrate, modification A

Similar as Example 2, but the wet material after filtration is dried at 40° C. and 12 mbar for 24 hours.

TGA analysis confirmed the product was 6-(6-hydroxymethyl-pyrimidin-4-yloxy)-naphthalene-1-carboxylic acid (3-trifluoromethyl-phenyl)-amide hemihydrate, Mod. A.

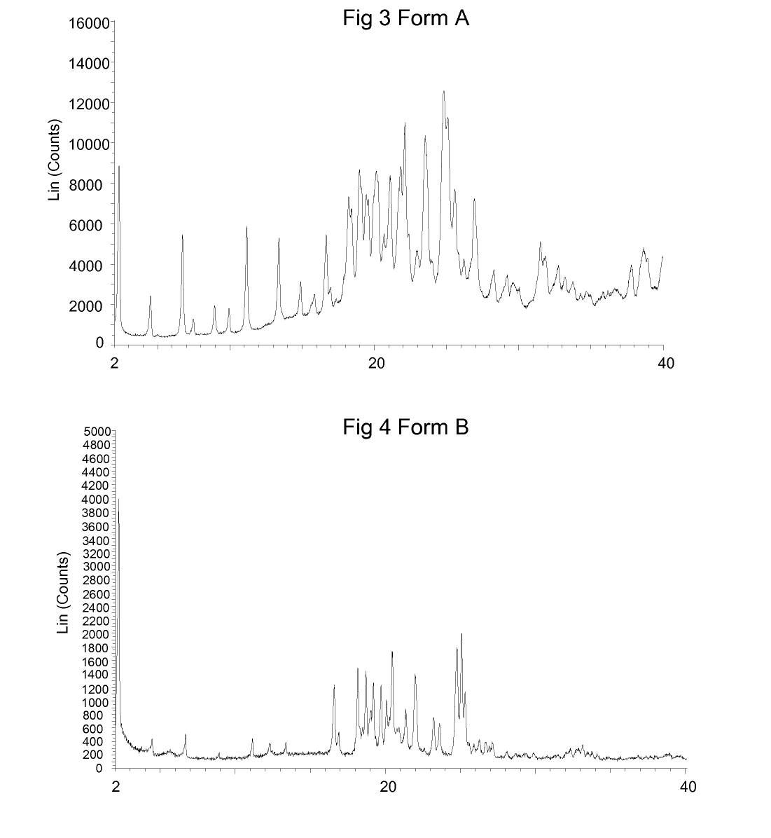

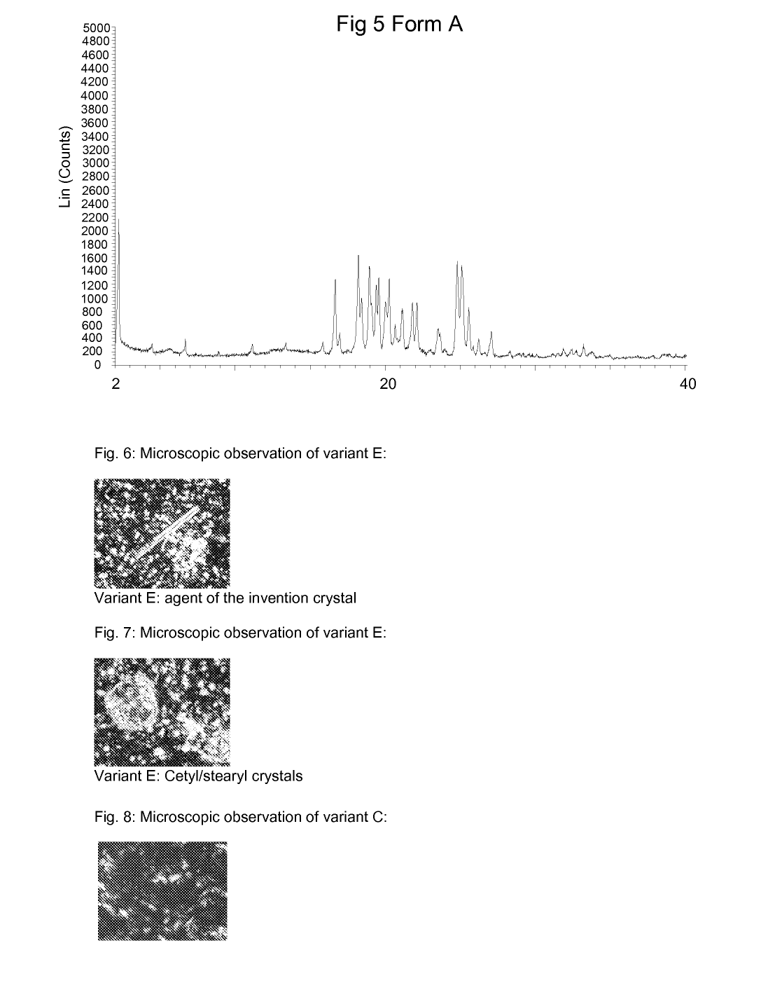

XRPD analysis was performed as described below; this also confirmed the product was 6-(6-hydroxymethyl-pyrimidin-4-yloxy)-naphthalene-1-carboxylic acid (3-trifluoromethyl-phenyl)-amide hemihydrate, Mod. A. FIG. 3 shows the obtained XRPD pattern in reflexion geometry; the background contribution is due to a kapton foil which is used to protect the sample. The instrument parameters were as follows: Bruker D8 Advance X-Ray diffractometer, Mode reflexion, Scan range 2°-40° (2 theta value), CuKα (45 kV, 40 mA). FIG. 5 shows the obtained XRPd pattern in transmission geometry. The instrument parameters were as follows: Bruker D8 Vario X-Ray diffractometer, Mode Transmission, Scan range 2°-40° (2 theta value), CuKα (45 kV, 40 mA). Temperature: 20 Degrees C.

### Example 5

### 6-(6-Hydroxymethyl-pyrimidin-4-yloxy)-naphthalene-1-carboxylic acid (3-trifluoromethyl-phenyl)-amide hemihydrate, modification A

Similar as Example 3, but the wet material after filtration is dried at 40° C. and 10 mbar for 24 hours. XRPD and TGA show the product is the over-dried 6-(6-hydroxymethyl-pyrimidin-4-yloxy)-naphthalene-1-carboxylic acid (3-trifluoromethyl-phenyl)-amide hemihydrate Mod. A.

### B Pharmaceutical Compositions, Solution Type

An ointment was prepared by combining the excipients as indicated below with the agent of the invention, specifically, all components as indicated below, except water, citric acid and HPC, were combined and heated to 65° C. to obtain a melt. Water and when applicable HPC, and citric acid were heated to 65° C. and added at this temperature to the obtained melt. The obtained composition was slowly cooled down to room temperature to obtain a composition of the solution type. The agent of the invention was obtained as described above.

| | Ointment | Ointment | Ointment | Ointment |
|---|---|---|---|---|
| | Var A [%] | Var B [%] | Var C [%] | Var E [%] |
| agent of invention | 0.8 | 0.8 | 0.8 | 1.0 |
| Polyethylene glycol | 55.0 | 55.0 | 55.0 | 55.0 |
| 400 PF | | | | |
| Polyethylene glycol | 25.0 | — | — | 25.0 |
| 3000 PH | | | | |
| Polyethylene glycol | — | — | 28.0 | — |
| 4000 | | | | |
| Polyethylene glycol | — | 28.0 | — | — |
| 6000 | | | | |
| Hydroxypropyl | 0.8 | — | — | — |
| cellulose | | | | |
| Benzoic acid | — | — | — | 0.15 |
| Stearyl alcohol | — | — | — | 1.0 |
| Cetyl alcohol | — | — | — | 1.0 |
| Butylhydroxy | 0.1 | 0.1 | 0.1 | — |
| toluene (BHT) | | | | |
| Citric acid | 0.07 | 0.07 | 0.07 | — |
| anhydrous | | | | |
| Water nanopure | 18.23 | 16.03 | 16.03 | 16.85 |

### C Pharmaceutical Compositions, Suspension Type

An ointment was prepared by combining the excipients as indicated below with the agent of the invention. Specifically, all components as indicated below, except for the agent of the invention, were combined and heated to 85° C. to obtain a melt. The obtained melt was cooled down to 70° C. The agent of the invention was heated to be added at this temperature. The obtained composition was slowly cooled down to room temperature to obtain a composition of the suspension type. The agent of the invention was obtained as described above.

| | ointment Var |
|---|---|
| | H [%] |
| agent of invention | 1 |
| liquid paraffin | 30 |
| (mineral oil) | |
| white Vaseline | 53.5 |
| (petrolatum) | |
| microcrystalline wax | 12.5 |
| (hydrocarbons) | |
| Isopropyl myristate | 3.0 |

### D Stability Tests and Scale-Up

The pharmaceutical compositions, solution type, as prepared above, were tested for chemical stability. After 13 weeks of storage at 40° C., only 1.5% degradation product is detected. The pharmaceutical compositions, suspension type, were tested for chemical stability. After 12 weeks of storage at 40° C., less than 1% degradation product is detected. The chemical stability of the compositions was found to be very good.

The pharmaceutical compositions, solution type, as prepared above in a 50-500 g scale, were tested for physical stability. No recrystallisation after 12 weeks of the agent of the invention for lab batches was observed.

Recrystallization of the agent of the invention at 5° C. and room temperature for batches prepared in a 5-25 kg scale after 6 weeks was observed. Recrystallisation of the agent of invention after 6 weeks of storage at different temperatures, for Variant E. FIG. 6 depicts a microscopic observation of variant E, showing a crystal of the agent of the invention. In addition non suitable cosmetic feeling (“sandy effect”) was observed when applying this formulation on the skin due to recrystallisation of the cetyl and stearyl alcohol. FIG. 7 depicts a microscopic observation of variant E, showing cetyl/stearyl crystals. Drug substance recrystallization can be avoided by reducing drug concentration to 0.8%. Unfavorable cosmetic properties, “sandy feeling”, caused by cetyl and stearyl alcohol can be avoided by the use of alternate excipients leading to increased viscosity. FIG. 8 depicts a microscopic observation of variant C, lacking the “sandy feeling”.

Inhomogeneity of the variant A for batches prepared in a 5-25 kg scale was observed during stability study after storage at 40° C., due to precipitation of hydroxypropyl cellulose (Handbook of pharmaceutical excipients: HPC is insoluble in hot water and precipitate as a highly swollen flock at a temperature between 40° C. and 45° C.).

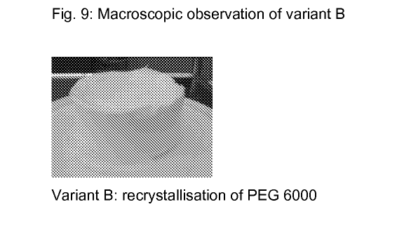

Up-scaling of the variant B to batches prepared in a 5-25 kg scale lead to re-crystallization/precipitation of PEG6000. FIG. 9 shows macroscopic observation of variant B, demonstrating recrystallisation of PEG 6000.

The pharmaceutical compositions, suspension type, as prepared above were tested for physical stability. No crystal growth over 12 weeks was observed and matrix structure remained unchanged at 5° C. and RT.

The physical stability of both, the solution type composition C and the suspension type composition H were found to be very good.

The pharmaceutical compositions, solution type, as prepared above, were tested for chemical stability. 6 months accelerated and real time stability data for Variant C and A indicated 2 years shelf life. The good stability of these compositions is due to addition of BHT.

| | Agent of | Degradation | Agent of | Degradation | Agent of | Degradation |
|---|---|---|---|---|---|---|
| Storage conditions/ | invention | products | invention | products | invention | products |
| relative humidity | [%] | [%] | [%] | [%] | [%] | [%] |
| (time) | (Variant A) | | (Variant C) | | (Variant E) | |
| Initial analysis | 99.4 | <0.1 | 99.0 | <0.1 | 97.4 | <0.1 |
| 5° C. (6 M) | 100.3 | <0.1 | 99.8 | <0.1 | 96.7 | <0.1 |
| 25° C./60% RH (6 M) | 99.0 | 0.8 | 98.8 | 0.8 | 95.2 | 0.7 |
| 40° C./75% RH (6 M) | 98.9 | 1.4 | 97.2 | 1.5 | 93.7 | 1.3 |

The pharmaceutical compositions, solution type, as prepared above, were tested for photostability. The tests, according to ICH conditions, showed 3.8% of degradation.

The pharmaceutical compositions, suspension type, as prepared above, were tested for photostability. The tests, according to ICH conditions,) showed 1.9% of degradation.

Under typical use conditions, the degradation observed is not considered an issue.

The chemical stability of the suspension type composition Variant H was found to be very good. The total amount of degradation products did not exceed 0.1% over a period of up to 12 months at temperatures of 5 C. In addition, excellent chemical stability in terms of active substance was found over a period of up to 12 months at temperatures up to 30° C. and up to 6 months at 40° C., respectively.

| | | |
|---|---|---|
| | Agent of invention | Degradation |
| Storage conditions/ | [%] Variant | products [%] |

| | |
|---|---|
| relative humidity (time) | (Variant H) |

| | | |
|---|---|---|
| Initial analysis | 99.3 | <0.1 |
| 5° C. (6 M) | 98.9 | <0.1 |
| 5° C. (9 M) | 101.2 | <0.1 |
| 5° C. (12 M) | 100.0 | <0.1 |
| 25° C./60% RH (6 M) | 98.9 | <0.1 |
| 25° C./60% RH (9 M) | 100.3 | <0.1 |
| 25° C./60% RH (12 M) | 99.3 | <0.1 |
| 30° C./65% RH (6 M) | 100.5 | <0.1 |
| 30° C./65% RH (9 M) | 99.4 | <0.1 |
| 30° C./65% RH (12 M) | 105.4 | <0.1 |
| 40° C./75% RH (6 M) | 105.7 | <0.1 |

### E In Vivo Tests

### 1) Skin Penetration

The pharmaceutical compositions, solution type, as prepared above, were applied to pigs (4 cm² assay): Small skin areas (4 cm²) were treated topically for different time intervals (0.5-8 hrs); the last test was 30 min before the animals were sacrificed. Skin flaps with the treated sites in the centre were then dissected and removed. The skin flaps were spread and heated on metal blocks placed on the test sites for 1 minute to induce separation of epidermis and dermis. The loosened epidermis was detached and removed. 1 mm thick dermal sheets were removed from the treated, de-epidermized area with a dermatome. From these sheets 6 mm punch biopsies were taken and analysed for test compound concentration by LC/MS. The procedure described was done with careful avoidance of contamination of the dermal samples with superficially attached test compound.

The following table provides AUC values of the agent of the invention in pig dermis when applied epicutaneously in the identified compositions (n=8)

| | Ointment | Ointment | Ointment | Ointment |
|---|---|---|---|---|
| Composition | Var A [%] | Var B [%] | Var C [%] | Var E [%] |
| AUC (0-8 h), | 1.5 | 3.1 | 1.2 | 1.1 |
| (μg * h/g) | | | | |

AUC means area under the curve, and is a well known term in clinical pharmacology. The AUC value is the total uptake of the agent. All the ointments, solution type, enable good penetration of the agent of the invention into the skin.

Var B enables good skin penetration levels.

Variant C, containing 0.8% of the agent of invention is bioequivalent to CSF variant E containing 1.0% of this same agent (1.2 and 1.1 μg*h/g AUC values for Var C and E respectively).

The pharmaceutical compositions, solution type variant E and suspension type variant H, as prepared above, were applied to pigs (4 cm² assay). The levels of the agent of the invention in pig dermis after epicutaneous application were compared. Both the solution type and suspension type formulations enable good penetration of the agent of the invention into the skin. In particular the suspension type formulation enables unexpectedly good skin penetration levels.

| | Skin level | Skin level | Maximum |
|---|---|---|---|
| | 1 h after | 4 h after | skin level |
| | application [μg/g] | application [μg/g] | detected [μg/g] |
| Ointment Var | 0.6 | 1.1 | 1.10 |
| E [%] (solution | | | |
| type) | | | |
| Ointment Var | 1.7 | 1.5 | 2.28 |
| H [%] | | | |
| (suspension | | | |
| type) | | | |

### 2) Inhibition of Angiogenesis and Vascular Permeability

Vascular endothelial growth factors (VEGFs) have angiogenic and vascular permeability-promoting activities in vitro and in vivo. We utilized inhibition of these major VEGFs-mediated responses to demonstrate antagonistic effects of topically applied agent of the invention in swine skin models. Young domestic pigs were used because pig skin is very similar to human skin in architecture and permeability for xenobiotics. Inhibition of dermal angiogenesis related to the agent of the invention was tested with an “matrigel” assay which was developed for studies in pigs; inhibition of vascular permeability in the skin with a modified Miles assay. With matrigel implants into which endothelial cells immigrate to form new vessels anti-angiogenic effects of applied test articles can be assessed in vivo; with the Miles assay, extravasation of Evans blue-labeled albumin is measured after VEGF-induced vascular leakage.

a) Matrigel Assay in Domestic Pigs

Method: Test areas on both paramedian ventral abdominal sides of 16-18 kg weighing domestic pigs were topically treated with 0.5% 6-(6-hydroxymethyl-pyrimidin-4-yloxy)-naphthalene-1-carboxylic acid (3-trifluoromethyl-phenyl)-amide or the vehicle (ethanol/propylene glycol 3/7) alone. Treatment was performed twice daily on days 1-4. On day 2, 100 μl matrigel, loaded with the angiogenic factors 200 ng/nl VEGF-165 and 40 U/ml heparin were injected intradermally at 10 different sites of the treated areas. On day 5, the animals were sacrificed and 8 mm punches from the injection sites collected, the subcutaneous fatty tissue removed, the hemorrhagic plugs carefully dissected and weighed. Thereafter, the samples were digested with dispase and single cell suspensions prepared. The isolated cells were stained with the endothelial cell marker CD31 or isotype controls and analyzed with FACS after gating for endothelial cells.

Results: Increase in weights of implanted matrigel plugs results from vessel formation and influx of blood. Excised plugs from vehicle-treated sites had a mean weight of 108 mg, whereas plugs from sites which have been treated epicutaneously with 0.5% 6-(6-hydroxymethyl-pyrimidin-4-yloxy)-naphthalene-1-carboxylic acid (3-trifluoromethyl-phenyl)-amide weighed 88 mg, by 19% less (Table 1). In CD31+ cell number (gated on endothelial cells) plugs from agent of the invention and vehicle-treated sites differed by 66%. Thus, topically applied agent of the invention inhibited neoangiogenesis, which was in the present setting mainly driven by VEGF added to the matrigel prior to implantation. VEGF is a key factor in regulating angiogenesis.

TABLE 1
Weight and cellularity of matrigel plugs implanted interadermally
in domestic pigs treated topically with 6-(6-hydroxymethyl-
pyrimidin-4-yloxy)-naphthalene-1-carboxylic acid (3-trifluoromethyl-
phenyl)-amide or vehicle

| Treatment of | Weight (mg) of | | Cell counts | |
|---|---|---|---|---|
| implantation sites | excised plugs | | (CD31+)/μl^{§} | |
| 0.5% 6-(6-hydroxymethyl- | 87.8 | (5.1)*** | 353 | [136]*** |
| pyrimidin-4-yloxy)- | | | | |
| naphthalene-1-carboxylic | | | | |
| acid (3-trifluoromethyl- | | | | |
| phenyl)-amide* | | | | |
| Vehicle | 107.9 | (28.3) | 818 | [121] |

*dissolved in ethanol:propylene glycol 3/7,
^{§}gated on endothelial cells,
***p < 0.001 vs vehicle controls, mean [SEM],
n: 15 (6-(6-hydroxymethyl-pyrimidin-4-yloxy)-naphthalene-1-carboxylic acid (3-trifluoromethyl-phenyl)-amide-sites) and 18 (vehicle)

b) Miles Assay in Domestic Pigs

Method: Test areas of 5×20 cm on both paramedian ventral abdominal sides of 16-18 kg weighing domestic pigs were topically treated with 1 ml of 0.8% 6-(6-hydroxymethyl-pyrimidin-4-yloxy)-naphthalene-1-carboxylic acid (3-trifluoromethyl-phenyl)-amide trice (30, 7 and 3 hrs prior to elicitation of vascular leakage with VEGF). 6-(6-Hydroxymethyl-pyrimidin-4-yloxy)-naphthalene-1-carboxylic acid (3-trifluoromethyl-phenyl)-amide was applied as composition, solution type, Variant C, as prepared above or dissolved in ethanol/propylene glycol (3/7). Control animals were treated similarly with the corresponding placebos (composition, solution type, Variant C, without 6-(6-hydroxymethyl-pyrimidin-4-yloxy)-naphthalene-1-carboxylic acid (3-trifluoromethyl-phenyl)-amide). Next, VEGF 165 (R&D Systems, 10 ng in 50 μl PBS) was injected at 4 sites on both treated areas. Ten minutes earlier, 2% Evans blue solution was injected intravenously (2 ml/kg body weight) to measure extravasation. Thirty minutes after the challenge with VEGF, the animals were killed and 8 mm punch biopsies taken from the injection sites. Evans blue was extracted from the biopsies with 0.5 ml formamide and the concentration of Evans blue in the supernatants was measured photometrically. Pre-tests have revealed that injection of PBS alone did not result in a measurable extravasation of Evans blue. Therefore, sites injected with PBS only were not included as controls.

Results: Pretreated of skin areas with composition, solution type, Variant C, inhibited VEGF-induced extravasation by 33% (p>0.01) compared to extravasation at sites treated with the composition, solution type, Variant C, without 6-(6-hydroxymethyl-pyrimidin-4-yloxy)-naphthalene-1-carboxylic acid (3-trifluoromethyl-phenyl)-amide (placebo). Application of agent of the invention dissolved in ethanol/propylene glycol caused inhibition by 24%. This data indicate that epicutaneously applied agent of the invention has penetrated in sufficient concentrations into the dermis to exhibit anti-VEGF activity.

TABLE 2
Evans blue concentration (as a measure of vascular leakage)
in VEGF-conditioned dermal tissue extracts from sites
treated with agent of the invention or placebo

| | Evans blue concentration |
|---|---|
| Pre-treatment of VEGF-injected test sites | (μg/ml tissue extract) |
| composition, solution type, Variant C | 1.67 (0.24) p > 0.001+ |
| 0.8% 6-(6-hydroxymethyl-pyrimidin-4-yloxy)- | 1.26 (0.13)++ |
| naphthalene-1-carboxylic acid (3- | |
| trifluoromethyl-phenyl)-amide* | |
| Placebo (inactive cream) | 1.12 (0.28)+ |

+Mean (SD) of 32 test sites in 3 animals;
++mean (SD) of 16 sites in 2 animals
***p < 0.001 vs placebo;
**p< 0.01 vs placebo
*dissolved in ethanol:propylene glycol 3/7

The results indicate that 6-(6-hydroxymethyl-pyrimidin-4-yloxy)-naphthalene-1-carboxylic acid (3-trifluoromethyl-phenyl)-amide, topically applied to skin and mucosal membranes, inhibit major VEGF-mediated effects, such as extravasation and angiogenesis. Increased vascular permeability occurs prior to new blood vessel formation. Therefore topical treatment with agent of the invention will be efficacious against diseases associated with vascular permeability and formation of vessels.

The following are also embodiments of the present invention:
A. A topical pharmaceutical composition containing 6-(6-Hydroxymethyl-pyrimidin-4-yloxy)-naphthalene-1-carboxylic acid (3-trifluoromethyl-phenyl)-amide or a solvate thereof and one or more pharmaceutically acceptable excipients; preferably a semi-solid, topical pharmaceutical composition.
B. A composition according to A containing
   a) 6-(6-Hydroxymethyl-pyrimidin-4-yloxy)-naphthalene-1-carboxylic acid (3-trifluoromethyl-phenyl)-amide or a solvate thereof; and
   b) a hydrophilic matrix, said matrix containing one or more types of polyethyleneglycol (PEG) and optionally water.
C. A composition according to B wherein the matrix b) contains low molecular PEG, high molecular PEG and optionally water; preferably a PEG having a molecular mass of 100-1000 g/mol, a PEG having a molecular mass of 2000-25000 g/mol and water.
D. A composition according to B or C wherein the matrix b) contains PEG having a molecular mass of 400 g/mol, PEG having a molecular mass of 6000 g/mol and water.
E. A composition according to any of B-D wherein component a) is present in an amount between 0.2-5 wt. % of the total composition and said matrix b) contains at least 50 wt. % PEG and at most 40 wt. % water.
F. A composition according to any of B-E further containing one or more excipients selected from the group consisting of antioxidants, gelling agents, ph adjusting agents/buffers, agents to modify consistency, preservatives, (co-) solvents, fillers, binders, disintegrators, flow conditioners, lubricants, fragrances, stabilizers, wetting agents, emulsifiers, solubilizers and salts for regulating osmotic pressure.
G. A composition according to any of B-F, which does not contain a penetration enhancer.
H. A composition according to A containing
   a) 6-(6-Hydroxymethyl-pyrimidin-4-yloxy)-naphthalene-1-carboxylic acid (3-trifluoromethyl-phenyl)-amide or a solvate thereof;
   b) a hydrophobic matrix; and
   c) a penetration enhancer.
I. A composition according to H wherein said matrix b) contains one or more compounds selected from the group consisting of paraffines, vegetable oils, animal fats, synthetic glycerides, waxes and liquid polysiloxanes.
J. A composition according to any of H or I, wherein said matrix b) contains least two types of hydrocarbons; preferably mineral oil, petrolatum, microcrystalline wax.
K. A composition according to any of H-J, wherein said penetration enhancer c) is selected from the group consisting of saturated fatty acids and esters thereof, particularly isoproylmyristate.
L. A composition according to any of H-K, wherein component a) is present in an amount between 0.2-5 wt. % of the total composition, component c) is present in an amount between 0.5-20 wt. % of the total composition and said matrix b) contains up to 66 wt. % mineral oil, up to 98 wt. % petrolatum, up to 25 wt. % microcrystalline wax.
M. A composition according to any of A-L for the treatment of, or for use in the treatment of, i) a dermatological disease or condition, ii) a disease, condition or damage of the retina, or iii) cosmetic dermatology, particularly psoriasis, atopic dermatitis, allergic contact dermatitis or rosacea.
N. A method of treatment of a dermatological disease or condition, which treatment comprises administering to a subject in need of such treatment, particularly a human, an effective amount of a composition as described herein.
O. A method of treatment of psoriasis, atopic dermatitis, allergic contact dermatitis or rosacea, which treatment comprises administering to a subject in need of such treatment, particularly a human, an effective amount of a composition as described herein.
P. 6-(6-Hydroxymethyl-pyrimidin-4-yloxy)-naphthalene-1-carboxylic acid (3-trifluoromethyl-phenyl)-amide in crystalline form.
Q. A compound of P in the form of a solvate, particularly the hemihydrate.
R. A compound of Q in the form of the hemihydrate characterized in that said compound comprises the following XRPd peaks (Modification B)

2-theta
12.3
16.6
16.9

or the following XRPd peaks (Modification A)

2-theta
15.8

or the following XRPd peaks (Modification A)

2-theta

7.4
9.9
14.9
15.8

S. A compound according to any of P-Q as a pharmaceutical.
T. A compound according to any of P-Q for the treatment of, or for use in the treatment of, i) a dermatological disease or condition, ii) a disease, condition or damage of the retina, iii) cosmetic dermatology; particularly for the treatment of, or for use in the treatment of, psoriasis, atopic dermatitis, allergic contact dermatitis or rosacea.

1. A topical pharmaceutical composition comprising 6-(6-hydroxymethyl-pyrimidin-4-yloxy)-naphthalene-1-carboxylic acid (3-trifluoromethyl-phenyl)-amide or a solvate thereof and a hydrophilic matrix, said matrix comprising one or more types of polyethyleneglycol (PEG) and optionally water.

2. A composition according to claim 1 wherein the matrix contains low molecular weight PEG having a molecular mass of 200-1000 g/mol, high molecular weight PEG having a molecular mass of 2000-5000 g/mol and optionally water.

3. A composition according to claim 1 wherein the matrix contains PEG having a molecular mass of 400 g/mol, PEG having a molecular mass of 4000 g/mol and water.

4. A composition according to claim 1 wherein said matrix contains between 10-80 wt. % low molecular weight PEG and between 10-80 wt. % high molecular weight PEG.

5. A composition according to claim 1 further containing 0.01%-2.0% of an antioxidant selected from the following excipients: butylated hydroxytoluene (BHT), butylated hydroxyanisole (BHA), alpha tocopherol, ascorbic acid or a mixture of thereof, butylated hydroxytoluene (BHT).

6. A composition according to claim 1 wherein 6-(6-hydroxymethyl-pyrimidin-4-yloxy)-naphthalene-1-carboxylic acid (3-trifluoromethyl-phenyl)-amide or a solvate thereof is present in an amount between 0.2-5 wt. % of the total composition and said matrix contains at least 50 wt. % PEG and at most 40 wt. % water.

7. A composition according to claim 1 further comprising one or more excipients selected from the group consisting of antioxidants, gelling agents, ph adjusting agents/buffers, agents to modify consistency, preservatives, (co-) solvents, fillers, binders, disintegrators, flow conditioners, lubricants, fragrances, stabilizers, wetting agents, emulsifiers, solubilizers and salts for regulating osmotic pressure.

8. A composition according to claim 1, which does not contain a penetration enhancer in amounts of at least 2.5 wt-%.

9. A topical pharmaceutical composition comprising 6-(6-hydroxymethyl-pyrimidin-4-yloxy)-naphthalene-1-carboxylic acid (3-trifluoromethyl-phenyl)-amide or a solvate thereof, a hydrophobic matrix; and a penetration enhancer.

10. A composition according to claim 9 wherein the matrix contains one or more compounds selected from the group consisting of paraffines, vegetable oils, animal fats, synthetic glycerides, waxes and liquid polysiloxanes.

11. A composition according to claim 9, wherein the matrix contains least two types of hydrocarbons.

12. A composition according to claim 9, wherein the penetration enhancer is selected from the group consisting of saturated fatty acids and esters thereof.

13. A composition according to claim 9, wherein 6-(6-hydroxymethyl-pyrimidin-4-yloxy)-naphthalene-1-carboxylic acid (3-trifluoromethyl-phenyl)-amide or a solvate thereof is present in an amount between 0.2-5 wt. % of the total composition, the penetration enhancer is present in an amount between 2.5-20 wt. % of the total composition and the matrix contains up to 66 wt. % mineral oil, up to 98 wt. % petrolatum, up to 25 wt. % microcrystalline wax.

14. (canceled)

15. (canceled)

16. A method of treatment of a dermatological disease or condition, which treatment comprises administering to a subject in need of such treatment, an effective amount of a composition according to claim 1.

17. A method of treatment of psoriasis, atopic dermatitis, allergic contact dermatitis or rosacea, which treatment comprises administering to a subject in need of such treatment, an effective amount of a composition according to claim 1.

18. 6-(6-Hydroxymethyl-pyrimidin-4-yloxy)-naphthalene-1-carboxylic acid (3-trifluoro-methyl-phenyl)-amide in crystalline form.

19. A compound of claim 18 in the form of a solvate, particularly the hemihydrate.

20. A compound of claim 19 in the form of hemihydrate characterized either by X-ray powder diffraction peaks at 7.4, 9.9, 14.9 and 15.8° 2-Theta for crystal form A or 2-theta values of 12.3, 16.6 and 16.9 in case of crystal form B.

21. (canceled)

22. (canceled)

23. (canceled)

24. (canceled)

25. A process for preparing a compound of formula (13), or a salt thereof, 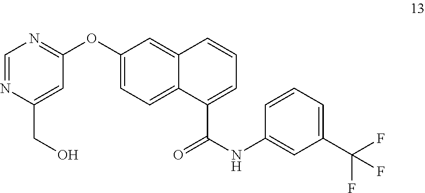
from a compound of formula (12), or salt thereof, 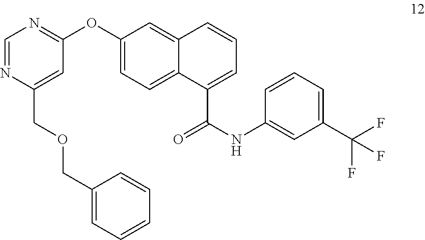
comprising
a) treating the compound of formula (12) with strong acid to obtain a compound of formula (13), optionally followed by
b) crystallizing the compound of formula (13), or salt thereof to obtain a compound of formula (14), 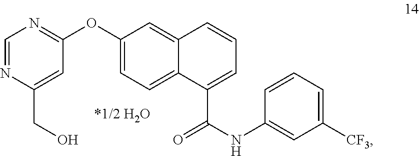
optionally followed by
c) milling the compound of formula (14).

26. A process according to claim 25 wherein a) and b) are performed as a one step procedure.

27. A process according to claim 25 wherein the strong acid is selected from trifluoroacetic acid or methanesulfonic acid.

28. A process for preparing a compound of formula (13), or a salt thereof, 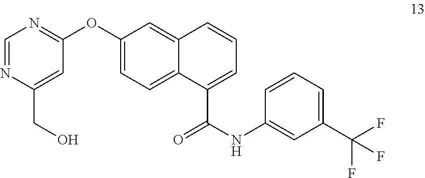
from a compound of formula (12), or salt thereof, 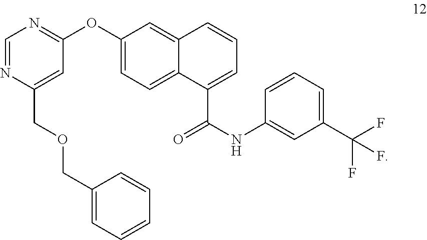
comprising
a) acylating the compound of formula (12) with an activating agent to obtain a compound of formula (15), or a salt thereof 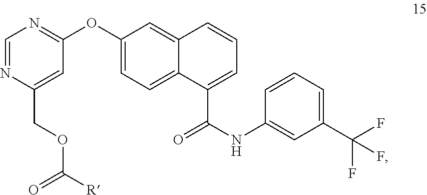
wherein R′ is selected from C₁-C₇-alkyl, followed by
b) deprotecting the compound of formula (15) with a suitable base to obtain a compound of formula (13), optionally followed by
c) crystallizing a compound of formula (13) to obtain a compound of formula (14) 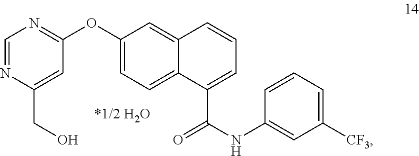
optionally followed by
d) milling the compound of formula (14).

29. A process according to claim 28 wherein b) and c) are performed as a one step procedure.

30. A process according claim 28 where in the acylating reagent is selected from acyl chlorides or acid anhydrides.

31. A process according claim 28 wherein the base is selected from sodium alkoxide, potassium alkoxide, sodium hydroxide, potassium hydroxide, sodium carbonate or potassium carbonate.

32. A process for preparing a compound of formula (12), or salt thereof, 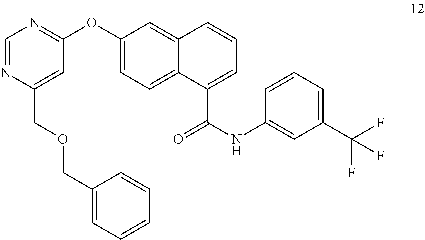
said process comprises
reacting a compound of compound of formula (5), or salt thereof, 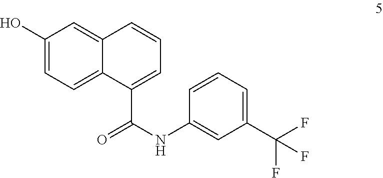
with the compound of formula (11) or salt thereof, 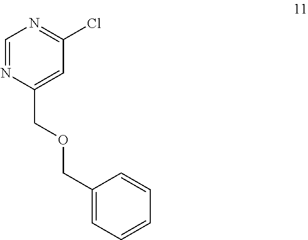
in the presence of a base.

33. A process according to claim 32 wherein the base is selected from potassium carbonate or cesium carbonate.

34. (canceled)

35. An intermediate of formula (15), or salt thereof, 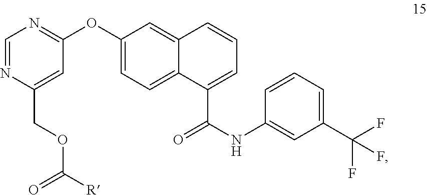
wherein R′ is selected from C₁-C₇-alkyl.

36. An intermediate of formula (12), or salt thereof, [chemical structure]
